(19) United States
(12) Patent Application Publication
(10) Pub. No.: US 2017/0151274 A1
(43) Pub. Date: Jun. 1, 2017

(54) COMPOSITIONS AND METHODS FOR ENHANCING IMMUNITY

(71) Applicant: Shaklee Corporation, Pleasanton, CA (US)

(72) Inventor: Hong WANG, Pleasanton, CA (US)

(21) Appl. No.: 15/317,837

(22) PCT Filed: Jul. 1, 2015
(86) PCT No.: PCT/US15/38889

§ 371 Date: Dec. 9, 2016

Related U.S. Application Data
(60) Provisional application No. 62/020,366, filed on Jul. 2, 2014.
(60) Provisional application No. 62/037,558, filed on Aug. 14, 2014.

(51) Int. Cl. A61K 31/716; A61K 36/07; A61K 36/258; A23P 10/00; A61K 9/20; A61K 9/48; A23L 2/52; A23L 33/10; A61K 36/064; A61K 9/00

(52) U.S. Cl. CPC A61K 31/716; A61K 36/064; A61K 36/07; A61K 36/258; A61K 9/0053; A61K 9/20; A61K 9/48; A61K 9/0056; A61K 9/0095; A23L 2/52; A23L 33/10; A23P 10/00; A23V 2002/00

(57) ABSTRACT

The present disclosure relates generally to immunostimulatory compositions, and more specifically to immunostimulatory compositions containing a β-glucan preparation, a ginseng extract, and a mushroom extract.

### CROSS-REFERENCE TO RELATED APPLICATION(S)

This application claims the benefit of U.S. Provisional Application No. 62/020,366, filed on Jul. 2, 2014, and of U.S. Provisional Application No. 62/037,558, filed on Aug. 14, 2014, each of which is incorporated herein by reference in its entirety.

### FIELD

The present disclosure relates generally to immunostimulatory compositions, and more specifically to immunostimulatory compositions containing a β-glucan preparation, a ginseng extract, and a mushroom extract.

### BACKGROUND

Immune systems are composed of multifaceted networks. In humans, the immune system is split into two main subgroups, the innate and adaptive immune system. The innate immune system is the first line of defense, rapidly responding to handle stresses and fight off invading viruses and microorganisms, while the adaptive immune system is involved in long term immunity, tasked with protecting the host against reinfection by foreign microbes. While these two systems are distinct in their roles in protecting the host, there exists extensive crosstalk between the two to promote general health. One way in which these systems communicate involves sending and receiving signals via protein messengers known as cytokines and chemokines. The signaling molecules involved, when in an appropriate balance, help the body to deal with stresses and maintain good health.

The use of herbal and natural medicines and remedies to promote health and wellness is becoming increasingly common. It is increasingly appreciated that at least some of the benefits afforded by natural remedies, including ginseng and mushroom extracts, are due to the immunostimulatory properties of polysaccharides, such as β-glucans, contained in these extracts (Curr Med Chem. 2000 July; 7(7):715-29; Chan et al., 2009, Journal of Hematology and Oncology 2: 25; Choi et al., 2008, Biosci. Biotechnol. Biochem 72: 7; Johnson et al., 2009, Scandanavian Journal of Immunology 69; Kim et al., 2011, Immune Network 11: 4). β-glucans, a type of polysaccharide consisting of chains of glucose of varying lengths, are found in various bacteria, yeast, fungi, and plants, and are known to be potent stimulators of the human immune system. It is thought that the secretion of various immune signaling molecules in response to β-glucan glucans is involved in the immune-enhancing properties of natural remedies.

To meet the increasing demand for natural remedies having increasingly beneficial immune-enhancing properties, there exists a need for additional and improved immunostimulatory compositions containing natural components.

### BRIEF SUMMARY

In one aspect, the present disclosure relates to an immunostimulatory composition containing a β-glucan preparation, a ginseng extract, and a mushroom extract; where the β-glucan glucan preparation is present in the range of about 40% to about 80% of the total dry weight of the β-glucan preparation, the ginseng extract, and the mushroom extract in the composition; where the ginseng extract is present in the range of about 10% to about 30% of the total dry weight of the β-glucan preparation, the ginseng extract, and the mushroom extract in the composition; and where the mushroom extract is present in the range of about 1% to about 20% of the total dry weight of the β-glucan preparation, the ginseng extract, and the mushroom extract in the composition. In some embodiments, the β-glucan preparation is prepared from yeast extract. In some embodiments, the yeast extract is obtained from Saccharomyces cerevisiae. In some embodiments that may be combined with any of the preceding embodiments, the β-glucan preparation is present at about 60% to about 80% of the total dry weight of the β-glucan preparation, the ginseng extract, and the mushroom extract. In some embodiments that may be combined with any of the preceding embodiments, the concentration of β-glucan in the β-glucan preparation is in the range of about 60% to about 80% of the total dry weight of the preparation. In some embodiments, the concentration of β-glucan in the β-glucan preparation is about 60% to about 75% of the total dry weight of the preparation. In some embodiments, the concentration of β-glucan in the β-glucan preparation is about 65% to about 70% of the total dry weight of the preparation. In some embodiments, the concentration of β-glucan in the β-glucan preparation is at least about 60%, about 65%, about 70%, or about 80% of the total dry weight of the preparation. In some embodiments that may be combined with any of the preceding embodiments, the ginseng extract is prepared from Chinese ginseng or American ginseng. In some embodiments that may be combined with any of the preceding embodiments, the ginseng extract is present at about 20% to about 30% of the total dry weight of the β-glucan preparation, the ginseng extract, and the mushroom extract. In some embodiments that may be combined with any of the preceding embodiments, the ginseng extract is present at about 20% to about 25% of the total dry weight of the β-glucan preparation, the ginseng extract, and the mushroom extract. In some embodiments that may be combined with any of the preceding embodiments, the ginseng extract is present at about 20%, about 25%, or about 30% of the total dry weight of the β-glucan preparation, the ginseng extract, and the mushroom extract. In some embodiments that may be combined with any of the preceding embodiments, the concentration of polysaccharides in the ginseng extract is about 5% to about 50% of the total dry weight of the ginseng extract. In some embodiments, the concentration of polysaccharides in the ginseng extract is about 10% to about 45% polysaccharides of the total dry weight of the ginseng extract. In some embodiments, the concentration of polysaccharides in the ginseng extract is about 20% to about 35% of total dry weight of the ginseng extract. In some embodiments that may be combined with any of the preceding embodiments, the mushroom extract is prepared from a mushroom selected from princess Matsutake mushroom, Reishi mushroom, shitake mushroom, Enokie mushroom, and pine mushroom. In some embodiments that may be combined with any of the preceding embodiments, the mushroom extract is present at about 5% to about 15% of the total dry weight of the β-glucan preparation, the ginseng extract, and the mushroom extract. In some embodiments that may be combined with any of the preceding embodiments, the mushroom extract is present at about 5% to about 10% of the total dry weight of the β-glucan preparation, the ginseng extract, and the mushroom extract. In some embodiments that may be combined with any of the preceding embodiments, the mushroom extract is about 5%, about 7.5%, about 10%, or about 15% of the total dry weight of the β-glucan preparation, the ginseng extract, and the mushroom extract. In some embodiments that may be combined with any of the preceding embodiments, the concentration of polysaccharides in the mushroom extract is about 30% to about 55% of the total dry weight of the mushroom extract. In some embodiments that may be combined with any of the preceding embodiments, the concentration of polysaccharides in the mushroom extract is about 30%, about 35%, about 40%, about 45%, about 50%, or about 55% of the total dry weight of the mushroom extract. In some embodiments that may be combined with any of the preceding embodiments, the β-glucan preparation is present at about 65% to about 70% of the total dry weight of the β-glucan preparation, the ginseng extract, and the mushroom extract; the ginseng extract is from Chinese ginseng and is present at about 20% to about 25% of the total dry weight of the β-glucan preparation, the ginseng extract, and the mushroom extract, and; the mushroom extract is from princess Matsutake mushroom and is present at about 5% to about 10% of the total dry weight of the β-glucan preparation, the ginseng extract, and the mushroom extract. In some embodiments that may be combined with any of the preceding embodiments, the β-glucan preparation is present at about 75% to about 80% of the total dry weight of the β-glucan preparation, the ginseng extract, and the mushroom extract; the ginseng extract is from Chinese ginseng and is present at about 15% to about 20% of the total dry weight of the β-glucan preparation, the ginseng extract, and the mushroom extract, and; the mushroom extract is from princess Matsutake mushroom and is present at about 1% to about 5% of the total dry weight of the β-glucan preparation, the ginseng extract, and the mushroom extract. In some embodiments that may be combined with any of the preceding embodiments, the β-glucan preparation is present at about 65% to about 70% of the total dry weight of the β-glucan glucan preparation, the ginseng extract, and the mushroom extract; the ginseng extract is from Chinese ginseng and is present at about 10% to about 15% of the total dry weight of the β-glucan preparation, the ginseng extract, and the mushroom extract, and; the mushroom extract is from princess Matsutake mushroom and is present at about 15% to about 20% of the total dry weight of the β-glucan preparation, the ginseng extract, and the mushroom extract. In some embodiments that may be combined with any of the preceding embodiments, the β-glucan preparation is present at about 65% to about 70% of the total dry weight of the β-glucan preparation, the ginseng extract, and the mushroom extract; the ginseng extract is from American ginseng and is present at about 20% to about 25% of the total dry weight of the β-glucan preparation, the ginseng extract, and the mushroom extract, and; the mushroom extract is from princess Matsutake mushroom and is present at about 5% to about 10% of the total dry weight of the β-glucan preparation, the ginseng extract, and the mushroom extract. In some embodiments that may be combined with any of the preceding embodiments, the β-glucan preparation is present at about 65% to about 70% of the total dry weight of the β-glucan preparation, the ginseng extract, and the mushroom extract; the ginseng extract is from Chinese ginseng and is present at about 20% to about 25% of the total dry weight of the β-glucan preparation, the ginseng extract, and the mushroom extract, and; the mushroom extract is from Reishi mushroom and is present at about 5% to about 10% of the total dry weight of the β-glucan preparation, the ginseng extract, and the mushroom extract. In some embodiments that may be combined with any of the preceding embodiments, the composition may comprise one or more additives described herein.

In another aspect, the present disclosure relates to an orally consumable product containing the composition of any one of the previous embodiments. In some embodiments, the orally consumable product is selected from a foodstuff composition, a beverage product, a dietary supplement, a nutraceutical, an edible gel mix, an edible gel composition, a granulated powder, a soft gel composition, a flash dissolve composition, and a pharmaceutical composition. In some embodiments, the orally consumable product is formulated into a tablet or capsule. In some embodiments, the orally consumable product is a foodstuff composition selected from a confectionary composition, a condiment, a cereal composition, a baked good, and a dairy product. In some embodiments, the orally consumable product is a carbonated or non-carbonated beverage product. In some embodiments, the orally consumable product is a beverage product selected from a soft drink, a fountain beverage, a frozen and ready-to-drink beverage, coffee, tea, a dairy beverage, a powdered soft drink, a liquid concentrate, fruit juice, a sport drink, and an energy drink. In some embodiments that may be combined with any of the preceding embodiments, the orally consumable product contains one or more additives selected from a carbohydrate, a polyol, an amino acid or salt thereof, a poly-amino acid or salt thereof, a sugar acid or salt thereof, a nucleotide, an organic acid, an inorganic acid, an organic salt, an organic acid salt, an organic base salt, an inorganic salt, a bitter compound, a flavorant, a flavoring ingredient, an astringent compound, a protein, a protein hydrolysate, a surfactant, an emulsifier, a flavonoid, an alcohol, a polymer, and combinations thereof.

In another aspect, the present disclosure relates to a method of preparing a composition, the method including combining a β-glucan preparation, a ginseng extract, and a mushroom extract into a composition, where the composition is formulated such that the β-glucan glucan preparation is present in the range of about 40% to about 80% of the total dry weight of the β-glucan preparation, the ginseng extract, and the mushroom extract in the composition, the ginseng extract is present in the range of about 10% to about 30% of the total dry weight of the β-glucan preparation, the ginseng extract, and the mushroom extract in the composition, and the mushroom extract is present in the range of about 1% to about 20% of the total dry weight of the β-glucan preparation, the ginseng extract, and the mushroom extract in the composition. In some embodiments, the β-glucan preparation is prepared from yeast extract. In some embodiments, the yeast extract is obtained from Saccharomyces cerevisiae. In some embodiments that may be combined with any of the preceding embodiments, the β-glucan preparation is present at about 60% to about 80% of the total dry weight of the β-glucan preparation, the ginseng extract, and the mushroom extract. In some embodiments that may be combined with any of the preceding embodiments, the β-glucan preparation is present at about 65% to about 70% of the total dry weight of the β-glucan preparation, the ginseng extract, and the mushroom extract. In some embodiments that may be combined with any of the preceding embodiments, the concentration of β-glucan in the β-glucan preparation is in the range of about 60% to about 80% of the total dry weight of the preparation. In some embodiments, the concentration of β-glucan in the β-glucan preparation is about 60% to about 75% of the total dry weight of the preparation. In some embodiments, the concentration of β-glucan in the β-glucan preparation is at least about 60%, about 65%, about 70%, or about 80% of the total dry weight of the preparation. In some embodiments that may be combined with any of the preceding embodiments, the ginseng extract is prepared from Chinese ginseng or American ginseng. In some embodiments that may be combined with any of the preceding embodiments, the ginseng extract is present at about 20% to about 30% of the total dry weight of the β-glucan preparation, the ginseng extract, and the mushroom extract. In some embodiments that may be combined with any of the preceding embodiments, the ginseng extract is present at about 20% to about 25% of the total dry weight of the β-glucan preparation, the ginseng extract, and the mushroom extract. In some embodiments that may be combined with any of the preceding embodiments, the ginseng extract is present at about 20%, about 25%, or about 30% of the total dry weight of the β-glucan preparation, the ginseng extract, and the mushroom extract. In some embodiments that may be combined with any of the preceding embodiments, the concentration of polysaccharides in the ginseng extract is about 5% to about 50% of the total dry weight of the ginseng extract. In some embodiments, the concentration of polysaccharides in the ginseng extract is about 10% to about 45% polysaccharides of the total dry weight of the ginseng extract. In some embodiments, the concentration of polysaccharides in the ginseng extract is about 20% to about 35% of total dry weight of the ginseng extract. In some embodiments that may be combined with any of the preceding embodiments, the mushroom extract is prepared from a mushroom selected from princess Matsutake mushroom, Reishi mushroom, shitake mushroom, Enokie mushroom, and pine mushroom. In some embodiments that may be combined with any of the preceding embodiments, the mushroom extract is present at about 5% to about 15% of the total dry weight of the β-glucan preparation, the ginseng extract, and the mushroom extract. In some embodiments that may be combined with any of the preceding embodiments, the mushroom extract is present at about 5% to about 10% of the β-glucan preparation, the ginseng extract, and the mushroom extract. In some embodiments that may be combined with any of the preceding embodiments, the mushroom extract is about 5%, about 7.5%, about 10%, or about 15% of the total dry weight of the β-glucan preparation, the ginseng extract, and the mushroom extract. In some embodiments that may be combined with any of the preceding embodiments, the concentration of polysaccharides in the mushroom extract is about 30% to about 55% of the total dry weight of the mushroom extract. In some embodiments that may be combined with any of the preceding embodiments, the concentration of polysaccharides in the mushroom extract is about 30%, about 35%, about 40%, about 45%, about 50%, or about 55% of the total dry weight of the mushroom extract. In some embodiments that may be combined with any of the preceding embodiments, the β-glucan preparation is present at about 65% to about 70% of the total dry weight of the β-glucan preparation, the ginseng extract, and the mushroom extract; the ginseng extract is from Chinese ginseng and is present at about 20% to about 25% of the total dry weight of the β-glucan preparation, the ginseng extract, and the mushroom extract, and; the mushroom extract is from princess Matsutake mushroom and is present at about 5% to about 10% of the total dry weight of the β-glucan preparation, the ginseng extract, and the mushroom extract. In some embodiments that may be combined with any of the preceding embodiments, the β-glucan preparation is present at about 75% to about 80% of the total dry weight of the β-glucan preparation, the ginseng extract, and the mushroom extract; the ginseng extract is from Chinese ginseng and is present at about 15% to about 20% of the total dry weight of the β-glucan preparation, the ginseng extract, and the mushroom extract, and; the mushroom extract is from princess Matsutake mushroom and is present at about 1% to about 5% of the total dry weight of the β-glucan preparation, the ginseng extract, and the mushroom extract. In some embodiments that may be combined with any of the preceding embodiments, the β-glucan preparation is present at about 65% to about 70% of the total dry weight of the β-glucan preparation, the ginseng extract, and the mushroom extract; the ginseng extract is from Chinese ginseng and is present at about 10% to about 15% of the total dry weight of the β-glucan preparation, the ginseng extract, and the mushroom extract, and; the mushroom extract is from princess Matsutake mushroom and is present at about 15% to about 20% of the total dry weight of the β-glucan glucan preparation, the ginseng extract, and the mushroom extract. In some embodiments that may be combined with any of the preceding embodiments, the β-glucan preparation is present at about 65% to about 70% of the total dry weight of the β-glucan preparation, the ginseng extract, and the mushroom extract; the ginseng extract is from American ginseng and is present at about 20% to about 25% of the total dry weight of the β-glucan preparation, the ginseng extract, and the mushroom extract, and; the mushroom extract is from princess Matsutake mushroom and is present at about 5% to about 10% of the total dry weight of the β-glucan preparation, the ginseng extract, and the mushroom extract. In some embodiments that may be combined with any of the preceding embodiments, the β-glucan preparation is present at about 65% to about 70% of the total dry weight of the β-glucan preparation, the ginseng extract, and the mushroom extract; the ginseng extract is from Chinese ginseng and is present at about 20% to about 25% of the total dry weight of the β-glucan preparation, the ginseng extract, and the mushroom extract, and; the mushroom extract is from Reishi mushroom and is present at about 5% to about 10% of the total dry weight of the β-glucan preparation, the ginseng extract, and the mushroom extract. In some embodiments that may be combined with any of the preceding embodiments, the method further includes combining one or more additives into the composition. In some embodiments that may be combined with any of the preceding embodiments, the method further includes preparing the composition into an orally consumable product.

In another aspect, the present disclosure relates to a method of enhancing an immune function in a subject, the method including administering to a subject a composition or product of any one of the preceding embodiments. In some embodiments, the subject is an animal. In some embodiments, the subject is a human. In some embodiments that may be combined with any of the preceding embodiments, the enhanced immune function includes enhanced lymphocyte proliferation and/or enhanced production of one or more cytokines. In some embodiments, the one or more cytokines are selected from IFN-γ, IL-12, and IL-10.

In another aspect, the present disclosure relates to a method of enhancing an immune function in a subject, the method including administering to a subject a composition including about 220 mg to about 250 mg of a β-glucan preparation, about 75 to about 90 mg of a ginseng extract, and about 20 mg to about 40 mg of a mushroom extract, where the composition is administered once daily. In some embodiments, the composition includes about 220 mg, about 230 mg, about 240 mg, or about 250 mg of a β-glucan preparation. In some embodiments that may be combined with any of the preceding embodiments, the composition includes about 80 mg, about 82 mg, about 84 mg, about 86 mg, about 88 mg, or about 90 mg of a ginseng extract. In some embodiments that may be combined with any of the preceding embodiments, the composition includes about 20 mg, about 25 mg, about 30 mg, about 33 mg, about 35 mg, about 38 mg, or about 40 mg of a mushroom extract. In some embodiments that may be combined with any of the preceding embodiments, the composition includes about 220 mg, about 230 mg, about 240 mg, or about 250 mg of a β-glucan preparation; about 80 mg, about 82 mg, about 84 mg, about 86 mg, about 88 mg, or about 90 mg of a ginseng extract; and about 20 mg, about 25 mg, about 30 mg, about 33 mg, about 35 mg, about 38 mg, or about 40 mg of a mushroom extract.

In another aspect, the present disclosure relates to a method of enhancing an immune function in a subject, the method including administering to a subject a composition including about 250 mg to about 325 mg of a β-glucan preparation, about 90 to about 120 mg of a ginseng extract, and about 30 mg to about 50 mg of a mushroom extract, where the composition is administered once daily. In some embodiments, the composition includes about 250 mg, about 265 mg, about 275 mg, about 290 mg, about 300 mg, about 310 mg, or about 325 mg of a β-glucan preparation. In some embodiments that may be combined with any of the preceding embodiments, the composition includes about 90 mg, about 95 mg, about 100 mg, about 105 mg, about 110 mg, about 115 mg, or about 120 mg of a ginseng extract. In some embodiments that may be combined with any of the preceding embodiments, the composition includes about 30 mg, about 35 mg, about 40 mg, about 45 mg, or about 50 mg of a mushroom extract. In some embodiments that may be combined with any of the preceding embodiments, the composition includes about 250 mg, about 265 mg, about 275 mg, about 290 mg, about 300 mg, about 310 mg, or about 325 mg of a β-glucan preparation; about 90 mg, about 95 mg, about 100 mg, about 105 mg, about 110 mg, about 115 mg, or about 120 mg of a ginseng extract of a ginseng extract; and about 30 mg, about 35 mg, about 40 mg, about 45 mg, or about 50 mg of a mushroom extract.

In another aspect, the present disclosure relates to a method of enhancing an immune function in a subject, the method including administering to a subject a composition including about 150 mg to about 200 mg β-glucans from a β-glucan preparation, about 5 mg to about 10 mg polysaccharides from a ginseng extract, and about 8 mg to about 16 mg polysaccharides from a mushroom extract, where the composition is administered once daily. In some embodiments, the composition includes about 150 mg β-glucans, about 175 mg β-glucans, or about 200 mg β-glucan glucans from a β-glucan preparation. In some embodiments that may be combined with any of the preceding embodiments, the composition includes about 5 mg, about 6 mg, about 7 mg, about 8 mg, about 9 mg, or about 10 mg polysaccharides from a ginseng extract. In some embodiments that may be combined with any of the preceding embodiments, the composition includes about 8 mg, about 9 mg, about 10 mg, about 11 mg, about 12 mg, about 13 mg, about 14 mg, about 15 mg, or about 16 mg polysaccharides from a mushroom extract. In some embodiments that may be combined with any of the preceding embodiments, the composition includes about 150 mg β-glucans, about 175 mg β-glucans, or about 200 mg β-glucans from a β-glucan preparation; about 5 mg, about 6 mg, about 7 mg, about 8 mg, about 9 mg, or about 10 mg polysaccharides from a ginseng extract; and about 8 mg, about 9 mg, about 10 mg, about 11 mg, about 12 mg, about 13 mg, about 14 mg, about 15 mg, or about 16 mg polysaccharides from a mushroom extract.

In another aspect, the present disclosure relates to a method of enhancing an immune function in a subject, the method including administering to a subject a composition including about 150 mg to about 200 mg β-glucans from a β-glucan preparation, about 20 mg to about 30 mg polysaccharides from a ginseng extract, and about 10 mg to about 20 mg polysaccharides from a mushroom extract, where the composition is administered once daily. In some embodiments, the composition includes about 150 mg β-glucans, about 175 mg β-glucans, or about 200 mg β-glucans from a β-glucan preparation. In some embodiments that may be combined with any of the preceding embodiments, the composition includes about 20 mg, about 25 mg, or about 30 mg polysaccharides from a ginseng extract. In some embodiments that may be combined with any of the preceding embodiments, the composition includes about 10 mg, about 12 mg, about 14 mg, about 16 mg, about 18 mg, or about 20 mg polysaccharides from a mushroom extract. In some embodiments that may be combined with any of the preceding embodiments, the composition includes about 150 mg β-glucans, about 175 mg β-glucans, or about 200 mg β-glucans from a β-glucan preparation; about 20 mg, about 25 mg, or about 30 mg polysaccharides from a ginseng extract; and about 10 mg, about 12 mg, about 14 mg, about 16 mg, about 18 mg, or about 20 mg polysaccharides from a mushroom extract.

In another aspect, the present disclosure relates to a method of enhancing an immune function in a subject, the method including administering to a subject a composition including about 175 mg to about 225 mg β-glucans from a β-glucan preparation, about 25 mg to about 45 mg polysaccharides from a ginseng extract, and about 15 mg to about 30 mg polysaccharides from a mushroom extract, where the composition is administered once daily. In some embodiments, the composition includes about 175 mg β-glucans, about 195 mg β-glucans, about 200 mg β-glucans, about 205 mg β-glucans, about 210 mg β-glucans, about 215 mg β-glucans, or about 225 mg β-glucans from a β-glucan preparation. In some embodiments that may be combined with any of the preceding embodiments, the composition includes about 25 mg, about 28 mg, about 30 mg, about 32 mg, about 35 mg, about 38 mg, about 40 mg, about 42 mg, or about 45 mg polysaccharides from a ginseng extract. In some embodiments that may be combined with any of the preceding embodiments, the composition includes about 15 mg, about 18 mg, about 20 mg, about 22 mg, about 24 mg, about 25 mg, or about 30 mg polysaccharides from a mushroom extract. In some embodiments that may be combined with any of the preceding embodiments, the composition includes about 175 mg β-glucans, about 195 mg β-glucan glucans, about 200 mg β-glucans, about 205 mg β-glucans, about 210 mg β-glucans, about 215 mg β-glucans, or about 225 mg β-glucans from a β-glucan preparation; about 25 mg, about 28 mg, about 30 mg, about 32 mg, about 35 mg, about 38 mg, about 40 mg, about 42 mg, or about 45 mg polysaccharides from a ginseng extract; and about 15 mg, about 18 mg, about 20 mg, about 22 mg, about 24 mg, about 25 mg, or about 30 mg polysaccharides from a mushroom extract.

In another aspect, the present disclosure relates to a kit for enhancing immune function in a subject, where the kit includes an immunostimulatory composition or product of any of the preceding embodiments. In some embodiments, the kit includes instructions for administering the immunostimulatory composition to the subject. In some embodiments that may be combined with any of the preceding embodiments, the immunostimulatory composition in the kit is present in a package.

In some embodiments that may be combined with any of the preceding embodiments, polysaccharide contents of the present disclosure are determined using the Anthrone reagent-based spectrophotometric methods as described herein (see Example 2). In some embodiments that may be combined with any of the preceding embodiments, β-glucan contents of the present disclosure are determined using the Food Chemicals Codex (FCC)-based assay as described herein.

### DESCRIPTION OF THE FIGURES

FIG. 1 illustrates GM-CSF protein levels in response to various concentrations of β-glucan glucan solution or “Blend A” solution.

FIG. 2A illustrates IL-8 protein levels in response to various concentrations of β-glucan glucan solution or “Blend A” solution. FIG. 2B illustrates TNF-α protein levels in response to various concentrations of β-glucan solution or “Blend A” solution.

FIG. 3A illustrates IL-1β protein levels in response to various concentrations of β-glucan glucan solution or “Blend A” solution. FIG. 3B illustrates IL-13 protein levels in response to various concentrations of β-glucan solution or “Blend A” solution.

FIG. 4A illustrates IL-6 protein levels in response to various concentrations of β-glucan glucan solution or “Blend A” solution. FIG. 4B illustrates IL-7 protein levels in response to various concentrations of β-glucan solution or “Blend A” solution.

FIG. 5A illustrates IL-12 protein levels in response to various concentrations of β-glucan glucan solution or “Blend A” solution. FIG. 5B illustrates IL-10 protein levels in response to various concentrations of β-glucan solution or “Blend A” solution.

FIG. 6A illustrates IFN-γ protein levels in response to various concentrations of β-glucan glucan solution or “Blend A” solution. FIG. 6B illustrates lymphocyte proliferation in response to various concentrations of β-glucan solution or “Blend A” solution.

FIG. 7A illustrates lymphocyte proliferation in response to various compositions. FIG. 7B illustrates GM-CSF protein levels in response to various compositions. FIG. 7C illustrates IFN-γ protein levels in response to various compositions. FIG. 7D illustrates IL-10 protein levels in response to various compositions.

FIG. 8A illustrates IL-8 protein levels in response to various compositions. FIG. 8B illustrates IL-10 protein levels in response to various compositions. FIG. 8C illustrates TNF-α protein levels in response to various compositions. FIG. 8D illustrates IL-6 protein levels in response to various compositions.

FIG. 9A illustrates IL-4 protein levels in response to various compositions. FIG. 9B illustrates IL-7 protein levels in response to various compositions. FIG. 9C illustrates IL-12 protein levels in response to various compositions.

FIG. 10A illustrates IL-5 protein levels in response to various compositions. FIG. 10B illustrates IL-2 protein levels in response to various compositions. FIG. 10C illustrates IL-13 protein levels in response to various compositions.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

The present disclosure relates generally to immunostimulatory compositions, and more specifically to immunostimulatory compositions containing a β-glucan preparation, a ginseng extract, and a mushroom extract.

The present disclosure is based, at least in part, on Applicants' discovery that a composition containing a β-glucan preparation, a ginseng extract, and a mushroom extract was surprisingly able to produce greater immune stimulation as compared to the immune stimulation observed for a composition containing purified β-glucans alone. Relative to the composition containing purified β-glucans alone, Applicant's immunostimulatory composition was able to elicit immune stimulation to a similar degree for IL-8, TNF-α, IL-1β, II-13, IL-6, and IL-7 and a greater degree for IL-12, IL-10, IFN-γ, and lymphocyte proliferation. This was observed even though Applicants' immunostimulatory composition contained fewer polysaccharides than the composition containing purified β-glucans alone, the polysaccharides having been thought to be the active immunostimulatory agents in the compositions.

Accordingly, disclosed herein are methods and compositions for enhancing immunity. The compositions of the present disclosure contain a β-glucan preparation, a ginseng extract, and a mushroom extract, and may be used in methods of enhancing immune function in a subject.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

It is understood that aspects and embodiments of the present disclosure described herein include “comprising,” “consisting,” and “consisting essentially of” aspects and embodiments.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

All references cited herein are hereby incorporated by reference in their entirety.

### β-Glucan Preparations

The compositions of the present disclosure contain a β-glucan preparation, where the β-glucan preparation contains β-glucans. A β-glucan preparation may be prepared, for example, by obtaining a source of β-glucans and extracting the β-glucans from the source. The extracted β-glucans may then be formulated into a β-glucan preparation such that the preparation contains substantially purified β-glucans. A β-glucan preparation may also take the form of a crude or partially purified extract from a source that contains β-glucans such as, for example, a yeast extract. A β-glucan preparation containing β-glucans may also be commercially obtained.

β-glucans are polysaccharides of D-glucose monomers linked by β-glycosidic bonds. β-glucans are structurally diverse molecules and can have varying degrees of branching. Various structures of β-glucans are known in the art and may be used in the compositions and methods of the present disclosure. In some embodiments, β-1,3/1,6 branched β-glucans are present in β-glucan glucan preparations.

β-glucans are present in a variety of organisms, including many bacteria, yeast, fungi, and plants. Various sources of β-glucans are known in the art and may be used in the compositions and methods of the present disclosure. Sources of β-glucans may include, for example, Glomerella cingulate, Grifola frondosa (Maitake mushroom), Lentinus (lentinula) edode (shiitake mushroom), Pneumocytis carinii, G. lucidum, Schizophyllum commune, Sclerotium rolfsii or S. glucanicum, S. sclerotiorum (ascomycotma), Candida albicans, Saccharomyces cerevisiae, Alcaligenes faecalis, Laminaria species (e.g. digitata), cereals such as Gramineae grasses, barley, oat, wheat, rye, rice, gram negative bacteria, and algae such as brown seaweeds. In some embodiments, the β-glucan preparation is prepared from yeast extract. In some embodiments, the yeast extract is obtained from Saccharomyces cerevisiae.

β-glucan preparations may be prepared using various methods well-known to those of skill in the art. For example, cellular material from a source of β-glucans can be obtained and the cellular material subjected to enzymolysis to lyse the cells and liberate structural components such as, for example, polysaccharides. The lysis extract may then undergo a separation step, followed by an acid-base treatment. The solution may then be centrifuged to collect a solution containing β-glucans, which may then be used in formulating a β-glucan preparation. β-glucan preparations may also be prepared, for example, according to the methods outlined by the Food Chemicals Codex (FCC). The FCC method provides guidance for obtaining β-glucans from yeast, and involves separation of the cell wall from yeast extract and treatment to remove lipids and proteins from the wall material. An acid treatment is then performed, followed by sterilization and pH adjustment, to obtain a substantially purified solution of β-glucans.

Various additional methods of preparing β-glucan preparations and quantifying the β-glucan glucans found therein are known in the art and include, for example, the GEM assay (Danielson et al., 2010, Journal of Agricultural and Food Chemistry 58:10305-10308). Briefly, the GEM assay involves contacting a β-glucan solution with KOH and the initial digestion with a lyticase, followed by incubation with an exo-1,3-β-D-glucanase and β-glucosidase mixture to convert β-glucan glucan in the sample to glucose. The converted glucose generated by the hydrolysis of the β-glucan glucans is then measured using an enzymatic method. The GEM assay is well-known in the art and may be used to measure both highly branched and unbranched β-1,3-glucans.

β-glucan preparations may contain various concentrations of substantially purified β-glucan glucan. β-glucan preparations may contain, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% β-glucans by total dry weight of the β-glucan preparation.

β-glucan preparations may contain, for example, about 1% to about 40%, about 10% to about 50%, about 20% to about 60%, about 30% to about 70%, about 40% to about 80%, about 50% to about 90%, or about 60% to about 100% β-glucans by total dry weight of the β-glucan glucan preparation. β-glucan preparations may contain, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 100% β-glucans by total dry weight of the β-glucan preparation.

β-glucan preparations may contain, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to about 100% β-glucan glucans by total dry weight of the β-glucan preparation.

In some embodiments, the concentration of β-glucan in the β-glucan preparation is in the range of about 60% to about 80% of the total dry weight of the preparation. In some embodiments, the concentration of β-glucan in the β-glucan preparation is in the range of about 60% to about 75% of the total dry weight of the preparation.

### Ginseng Extracts

The compositions of the present disclosure contain a ginseng extract. A ginseng extract may be prepared, for example, by obtaining a source of ginseng and extracting cellular contents from the ginseng source. Ginseng extracts may be prepared from an entire ginseng plant, or they may be prepared from specific tissues or regions such as, for example, ginseng root. The process of preparing extracts will lyse cellular material and liberate structural components from the cells such as, for example, polysaccharides. Ginseng extracts may also be obtained commercially.

Ginseng is a type of plant belonging to the genus Panax of the family Araliaceae. There are various species of ginseng, and these plants typically have fleshy roots. Various ginseng plants are known in the art and may be used in the compositions and methods of the present disclosure. The source of ginseng may be, for example, American ginseng or Chinese ginseng. In some embodiments, the ginseng extract of the present disclosure is obtained from Chinese ginseng (Panax ginseng C A Meyer).

Ginseng extracts may be prepared using various methods well-known to those of skill in the art. For example, a flow process involving the following steps may be used to prepare ginseng extract: ginseng root (0.5 to ˜1 cm)→extraction/tissue grinding→filtration→concentration→alcohol precipitation→separation→precipitation→spray drying to final extract product. Once a final extract product is obtained, the extract may be further purified twice using boiling water.

Ginseng extracts may contain various concentrations of polysaccharide. Ginseng extracts may contain, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% polysaccharides by total dry weight of the extract.

Ginseng extracts may contain, for example, about 1% to about 40%, about 10% to about 50%, about 20% to about 60%, about 30% to about 70%, about 40% to about 80%, about 50% to about 90%, or about 60% to about 100% polysaccharides by total dry weight of the extract. Ginseng extracts may contain, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 100% polysaccharides by total dry weight of the extract.

Ginseng extracts may contain, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to about 100% polysaccharides by total dry weight of the extract.

In some embodiments, the ginseng extract contains about 1% to about 75% polysaccharides by total dry weight of the extract. In some embodiments, the ginseng extract contains about 5% to about 50% polysaccharides by total dry weight of the extract. In some embodiments, the ginseng extract contains about 30% to about 60% polysaccharides by total dry weight of the extract. In some embodiments, the ginseng extract contains about 30% polysaccharides by total dry weight of the extract.

### Mushroom Extracts

The compositions of the present disclosure contain a mushroom extract. A mushroom extract may be prepared, for example, by obtaining a source of mushroom and extracting cellular contents from the mushroom source. Mushroom extracts may be prepared from an entire mushroom, or they may be prepared from specific tissues or regions such as, for example, the stalk (stipe) or the cap. The process of preparing extracts will lyse cellular material and liberate structural components from the cells such as, for example, polysaccharides. Mushroom extracts may also be obtained commercially.

Mushrooms are the fleshy fruiting bodies of many fungi. Fungi are taxonomically organized under Kingdom Fungi and include various phyla such as Ascomycota, Basidiomycota, Chytridiomycota, Glomeromycota, Microsporidia, and Zygomycota. Various mushrooms are known in the art and may be used in the compositions and methods of the present disclosure. The source of mushroom may be, for example, princess Matsutake mushroom, Reishi mushroom, shitake mushroom, Enokie mushroom, and pine mushroom. In some embodiments, the mushroom extract of the present disclosure is obtained from princess Matsutake mushroom (Agaricus blazei Murill). In some embodiments, the mushroom extract of the present disclosure is obtained from Reishi mushroom (Ganoderma lucidum).

Mushroom extracts may be prepared using various methods well-known to those of skill in the art. For example, a flow process involving the following steps may be used to prepare a mushroom extract: raw mushroom material extraction/tissue grinding decoction extract filtration collection of filtrate liquid concentration alcohol addition spray drying to final extract product. The raw mushroom material may come from a solid state fermentation base of mushroom mycelium. A decoction extract may be prepared using purified water. The filtration step should be used to obtain liquid filtrate. The concentration step may involve a vacuum and low temperature. The addition of alcohol may be performed to separate impurities from the extract. The spray drying step may be performed by adding purified water to the filtered extract, dissolving at 90° C., and finally spray drying to form a powdered extract.

Mushroom extracts may contain various concentrations of polysaccharide. Mushroom extracts may contain, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% polysaccharides by total dry weight of the extract.

Mushroom extracts may contain, for example, about 1% to about 40%, about 10% to about 50%, about 20% to about 60%, about 30% to about 70%, about 40% to about 80%, about 50% to about 90%, or about 60% to about 100% polysaccharides by total dry weight of the extract. Mushroom extracts may contain, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 100% polysaccharides by total dry weight of the extract.

Mushroom extracts may contain, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to about 100% polysaccharides by total dry weight of the extract.

In some embodiments, the mushroom extract contains about 1% to about 75% polysaccharides by total dry weight of the extract. In some embodiments, the mushroom extract contains about 5% to about 50% polysaccharides by total dry weight of the extract. In some embodiments, the mushroom extract contains about 40% to about 55% polysaccharides by total dry weight of the extract.

### Astragalus Root Extract

In some embodiments, the compositions of the present disclosure contain an Astragalus root extract. An Astragalus root extract may be prepared, for example, by obtaining a source of Astragalus root and extracting cellular contents from the Astragalus root source. The process of preparing extracts will lyse cellular material and liberate structural components from the cells such as, for example, polysaccharides.

Astragalus is a large genus of plants belonging to the legume family Fabaceae and the subfamily Faboideae. Various Astragalus plants are known in the art and may be used in the compositions and methods of the present disclosure.

Astragalus root extracts may be prepared using various methods well-known to those of skill in the art.

Astragalus root extracts may contain various concentrations of polysaccharide. Astragalus root extracts may contain, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% polysaccharides by total dry weight of the extract.

Astragalus root extracts may contain, for example, about 1% to about 40%, about 10% to about 50%, about 20% to about 60%, about 30% to about 70%, about 40% to about 80%, about 50% to about 90%, or about 60% to about 100% polysaccharides by total dry weight of the extract. Astragalus root extracts may contain, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 100% polysaccharides by total dry weight of the extract. In some embodiments, the Astragalus root extract contains about 50% to about 80% polysaccharides by total dry weight of the extract.

Astragalus root extracts may contain, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to about 100% polysaccharides by total dry weight of the extract.

In some embodiments, the Astragalus root extract contains about 1% to about 75% polysaccharides by total dry weight of the extract. In some embodiments, the Astragalus root extract contains about 5% to about 50% polysaccharides by total dry weight of the extract.

### Analysis of Preparations and Extracts

The methods of the present disclosure may involve a quality control analysis of the various preparations and extracts described herein. Quality control analysis may be performed to determine the identity and/or concentration of certain compounds in the preparations and extracts such as, for example, the concentration of polysaccharides in the preparations and extracts. Information regarding concentrations and/or identities of various compounds, such as polysaccharides, may be used to guide the methods of formulating the compositions of the present disclosure.

Methods of determining polysaccharide content in a sample are well-known in the art and are described herein. For example, a spectrophotometric method using UV/VIS may be used to determine polysaccharide content in a sample. In a particular assay, sugars react with a reagent called anthrone reagent (Morris et al., 1948, Science: 107) under acidic conditions to yield a blue-green color. The intensity of the color is measured spectrophotometrically using Dextran as a standard. See also Dreywood, 1946, Ind. Eng. Chem. (Anal. Ed.): 18, 499. See also Example 2 for a more detailed analysis of this procedure.

Methods of quantifying polysaccharides in a sample may involve other spectrophotometric-based methods. For example, a phenol-sulfuric acid spectrophotometry-based assay of crude polysaccharide may be used to quantify polysaccharide content in a sample. After precipitation separation of the polysaccharide-containing sample by ethanol and elimination of other soluble saccharides and impurities, polysaccharides react with phenol-sulfuric acid and produce a compound with an orange-red color. The degree of the orange-red coloration in the sample is directly proportional to the concentration of polysaccharides in the sample. Analysis of the sample under of wave length of 485 nm may be used to quantify the coloration and thus the polysaccharide content of the sample.

### Immunostimulatory Compositions

The immunostimulatory compositions of the present disclosure contain a β-glucan preparation, a ginseng extract, and a mushroom extract. In some embodiments, the immunostimulatory compositions of the present disclosure further contain an Astragalus root extract in addition to a β-glucan preparation, a ginseng extract, and a mushroom extract. In some embodiments, the compositions may include one or more additives as disclosed herein. The immunostimulatory compositions of the present disclosure may be used in methods of enhancing immune function in a subject.

The present disclosure also relates to immunostimulatory compositions for use in enhancing immune function in a subject.

The present disclosure also relates to use of an immunostimulatory composition for manufacture of an orally consumable product for enhancing immune function in a subject.

Immunostimulatory compositions of the present disclosure may contain one or more additives. The one or more additives may be present to add or enhance one or more characteristics of the orally consumable product, such as flavor, texture, aroma, color, shelf-life, etc. The one or more additives may already be present in the orally consumable product or may be added to the orally consumable product, or one or more compounds or ingredients used to make the orally consumable product. The orally consumable product may contain any suitable additive known in the art. Examples of suitable additives include, for example, carbohydrates, polyols, amino acids or salts thereof, poly-amino acids or salt thereof, sugar acids or salts thereof, nucleotides, organic acids, inorganic acids, organic salts, organic acid salts, organic base salts, inorganic salts, bitter compounds, flavorants, flavoring ingredients, astringent compounds, proteins, protein hydrolysates, surfactants, emulsifiers, flavonoids, alcohols, polymers, preservatives, thickening agents, food colorings, and combinations thereof.

In some embodiments of an immunostimulatory composition containing a β-glucan preparation, a ginseng extract, and a mushroom extract of the present disclosure, the β-glucan preparation is present at about 70% of the total dry weight of the β-glucan preparation, the ginseng extract, and the mushroom extract. In some embodiments of an immunostimulatory composition containing a β-glucan preparation, a ginseng extract, and a mushroom extract of the present disclosure, the ginseng extract is present at about 25% of the total dry weight of the β-glucan glucan preparation, the ginseng extract, and the mushroom extract. In some embodiments of an immunostimulatory composition containing a β-glucan preparation, a ginseng extract, and a mushroom extract of the present disclosure, the mushroom extract is present at about 10% of the total dry weight of the β-glucan preparation, the ginseng extract, and the mushroom extract. In some embodiments of an immunostimulatory composition containing a β-glucan preparation, a ginseng extract, and a mushroom extract of the present disclosure, the β-glucan preparation is present at about 70% of the total dry weight of the β-glucan preparation, the ginseng extract, and the mushroom extract; the ginseng extract is present at about 25% of the total dry weight of the β-glucan preparation, the ginseng extract, and the mushroom extract; and, the mushroom extract is present at about 10% of the total dry weight of the β-glucan preparation, the ginseng extract, and the mushroom extract.

In some embodiments of an immunostimulatory composition containing a β-glucan preparation, a ginseng extract, and a mushroom extract of the present disclosure, the β-glucan preparation is present at about 10% of the total dry weight of the composition. In some embodiments of an immunostimulatory composition containing a β-glucan preparation, a ginseng extract, and a mushroom extract of the present disclosure, the ginseng extract is present at about 3.5% of the total dry weight of the composition. In some embodiments of an immunostimulatory composition containing a β-glucan preparation, a ginseng extract, and a mushroom extract of the present disclosure, the mushroom extract is present at about 1.5% of the total dry weight of the composition. In some embodiments of an immunostimulatory composition containing a β-glucan preparation, a ginseng extract, and a mushroom extract of the present disclosure, the β-glucan preparation is present at about 10% of the total dry weight of the composition, the ginseng extract is present at about 3.5% of the total dry weight of the composition, and the mushroom extract is present at about 1.5% of the total dry weight of the composition.

In some embodiments, immunostimulatory compositions of the present disclosure do not contain one or more of mung bean, soybean, red date, scallion, garlic, lentil bean, leek, hawthorn fruit, onion, angelica root, licorice, dandelion root, Senegal root, ginger, olive, sesame seed, parsley, extracts thereof, or preparations thereof.

In some embodiments, immunostimulatory compositions of the present disclosure do not contain one or more of Cynara scolymus (artichoke), Uncaria tomentosa (Cat's claw), Paullinia cupana (guarana), Croton lechleri (Sangre de Grado), Malpighia glabra (acerola), Ylex paraguayiensis (Yerba mate), Stevia rebaudiana (stevia), Ginkgo biloba, Tabebuia impetiginosa (Pau d′arco), Echinacea purpurea, Peumus boldus (boldo), Aloe vera (aloe), Cistanche salsa, Cistanche deserticola (and other Cistanche sp.), Nopal opuntia (Prickly pear cactus), Citrus sinensis (Citrus aurantium) and other members of the citrus family (lemon, lime, tangerine, grapefruit), Camelia sinensis (tea), Plantago psyllium (psyllium), Amaranth edulis and other amaranth sp. (amaranth), Commiphora mukul (guggul lipid), Serenoa repens, Serenoa serrulata (saw palmetto), oat bran, rice bran, linseed, garlic, Ceratonia siliqua (locust been gum or flour from the seeds of carob tree), Cyanopsis tetragonoloba (guar gum, EU Food additive code E412), Xanthomonas campestris (xanthan gum), extracts thereof, and preparations thereof.

β-Glucan Preparation

The immunostimulatory compositions of the present disclosure may contain various concentrations of a β-glucan preparation. The β-glucan preparation may be present in the amount of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the β-glucan preparation, the ginseng extract, and the mushroom extract. In some embodiments, the β-glucan preparation is present at about 70% of the total dry weight of the β-glucan preparation, the ginseng extract, and the mushroom extract.

The β-glucan preparation may be present in compositions in the amount of, for example, about 1% to about 40%, about 10% to about 50%, about 20% to about 60%, about 30% to about 70%, about 40% to about 80%, about 50% to about 90%, or about 60% to about 95% of the total dry weight of the β-glucan preparation, the ginseng extract, and the mushroom extract. The β-glucan preparation may be present in compositions in the amount of, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the β-glucan preparation, the ginseng extract, and the mushroom extract.

The β-glucan preparation may be present in compositions in the amount of, for example about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the β-glucan preparation, the ginseng extract, and the mushroom extract.

The immunostimulatory compositions of the present disclosure may contain various concentrations of a β-glucan preparation in relation to the total dry weight of the composition. The concentration of the β-glucan preparation in the composition may be, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the composition. In some embodiments, the concentration of the β-glucan preparation in the composition is at least about 50% of the total dry weight of the composition. In some embodiments, the concentration of the β-glucan preparation in the composition is at least about 65% of the total dry weight of the composition. In some embodiments, the concentration of the β-glucan preparation in the composition is at least about 70% of the total dry weight of the composition. In some embodiments, the concentration of the β-glucan preparation in the composition is at least about 10% of the total dry weight of the composition.

The concentration of the β-glucan preparation in the composition may be, for example, about 1% to about 40%, about 10% to about 50%, about 20% to about 60%, about 30% to about 70%, about 40% to about 80%, about 50% to about 90%, or about 60% to about 95% of the total dry weight of the composition. The concentration of the β-glucan preparation in the composition may be, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the composition.

The concentration of the β-glucan preparation in the composition may be, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the composition.

The immunostimulatory compositions of the present disclosure may contain various concentrations of β-glucan from the β-glucan preparation. The concentration of β-glucan from the β-glucan preparation in the composition may be, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the composition. In some embodiments, the concentration of β-glucan from the β-glucan preparation in the composition is at least about 5% of the total dry weight of the composition. In some embodiments, the concentration of β-glucan from the β-glucan preparation in the composition is at least about 50% of the total dry weight of the composition.

The concentration of β-glucan from the β-glucan preparation in the composition may be, for example, about 1% to about 40%, about 10% to about 50%, about 20% to about 60%, about 30% to about 70%, about 40% to about 80%, about 50% to about 90%, or about 60% to about 95% of the total dry weight of the composition. The concentration of β-glucan from the β-glucan glucan preparation in the composition may be, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the composition.

The concentration of β-glucan from the β-glucan preparation in the composition may be, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the composition.

The contribution of β-glucan from the β-glucan preparation to the total dry weight of the polysaccharide content in the immunostimulatory compositions may vary. The concentration of β-glucan from the β-glucan preparation in the composition may be, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the polysaccharide content. In some embodiments, the concentration of β-glucan from the β-glucan preparation in the composition is about 80% of the total dry weight of the polysaccharide content.

The concentration of β-glucan from the β-glucan preparation in the composition may be, for example, about 1% to about 40%, about 10% to about 50%, about 20% to about 60%, about 30% to about 70%, about 40% to about 80%, about 50% to about 90%, or about 60% to about 95% of the total dry weight of the polysaccharide content. The concentration of β-glucan from the β-glucan preparation in the composition may be, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the polysaccharide content.

The concentration of β-glucan from the β-glucan preparation in the composition may be, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the polysaccharide content.

Ginseng Extract

The immunostimulatory compositions of the present disclosure may contain various concentrations of a ginseng extract. The ginseng extract may be present in the amount of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the β-glucan preparation, the ginseng extract, and the mushroom extract. In some embodiments, the ginseng extract is present at about 25% of the total dry weight of the β-glucan preparation, the ginseng extract, and the mushroom extract.

The ginseng extract may be present in compositions in the amount of, for example, about 1% to about 40%, about 10% to about 50%, about 20% to about 60%, about 30% to about 70%, about 40% to about 80%, about 50% to about 90%, or about 60% to about 95% of the total dry weight of the β-glucan preparation, the ginseng extract, and the mushroom extract. The ginseng extract may be present in compositions in the amount of, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the β-glucan preparation, the ginseng extract, and the mushroom extract.

The ginseng extract may be present in compositions in the amount of, for example about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the β-glucan preparation, the ginseng extract, and the mushroom extract.

The immunostimulatory compositions of the present disclosure may contain various concentrations of ginseng extract in relation to the total dry weight of the composition. The concentration of ginseng extract in the composition may be, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the composition. In some embodiments, the concentration of ginseng extract in the composition is at least about 25% of the total dry weight of the composition.

The concentration of ginseng extract in the composition may be, for example, about 1% to about 40%, about 10% to about 50%, about 20% to about 60%, about 30% to about 70%, about 40% to about 80%, about 50% to about 90%, or about 60% to about 95% of the total dry weight of the composition. The concentration of ginseng extract in the composition may be, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the composition.

The concentration of ginseng extract in the composition may be, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the composition.

The immunostimulatory compositions of the present disclosure may contain various concentrations of polysaccharides from the ginseng extract. The concentration of polysaccharides from the ginseng extract in the composition may be, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the composition. In some embodiments, the concentration of polysaccharides from the ginseng extract in the composition is at least about 8% of the total dry weight of the composition.

The concentration of polysaccharides from the ginseng extract in the composition may be, for example, about 1% to about 40%, about 10% to about 50%, about 20% to about 60%, about 30% to about 70%, about 40% to about 80%, about 50% to about 90%, or about 60% to about 95% of the total dry weight of the composition. The concentration of polysaccharides from the ginseng extract in the composition may be, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the composition.

The concentration of polysaccharides from the ginseng extract in the composition may be, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the composition.

The contribution of polysaccharides from the ginseng extract to the total dry weight of the polysaccharide content in the immunostimulatory compositions may vary. The concentration of polysaccharides from the ginseng extract in the composition may be, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the polysaccharide content. In some embodiments, the concentration of polysaccharides from the ginseng extract in the composition is about 13% of the total dry weight of the polysaccharide content.

The concentration of polysaccharides from the ginseng extract in the composition may be, for example, about 1% to about 40%, about 10% to about 50%, about 20% to about 60%, about 30% to about 70%, about 40% to about 80%, about 50% to about 90%, or about 60% to about 95% of the total dry weight of the polysaccharide content. The concentration of polysaccharides from the ginseng extract in the composition may be, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the polysaccharide content.

The concentration of polysaccharides from the ginseng extract in the composition may be, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the polysaccharide content.

Mushroom Extract

The immunostimulatory compositions of the present disclosure may contain various concentrations of a mushroom extract. The mushroom extract may be present in the amount of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the β-glucan preparation, the ginseng extract, and the mushroom extract. In some embodiments, the mushroom extract is present at about 10% of the total dry weight of the β-glucan preparation, the ginseng extract, and the mushroom extract.

The mushroom extract may be present in compositions in the amount of, for example, about 1% to about 40%, about 10% to about 50%, about 20% to about 60%, about 30% to about 70%, about 40% to about 80%, about 50% to about 90%, or about 60% to about 95% of the total dry weight of the β-glucan preparation, the ginseng extract, and the mushroom extract. The mushroom extract may be present in compositions in the amount of, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the β-glucan preparation, the ginseng extract, and the mushroom extract.

The mushroom extract may be present in compositions in the amount of, for example about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the β-glucan preparation, the ginseng extract, and the mushroom extract.

The immunostimulatory compositions of the present disclosure may contain various concentrations of mushroom extract in relation to the total dry weight of the composition. The concentration of mushroom extract in the composition may be, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the composition. In some embodiments, the concentration of mushroom extract in the composition is at least about 10% of the total dry weight of the composition.

The concentration of mushroom extract in the composition may be, for example, about 1% to about 40%, about 10% to about 50%, about 20% to about 60%, about 30% to about 70%, about 40% to about 80%, about 50% to about 90%, or about 60% to about 95% of the total dry weight of the composition. The concentration of mushroom extract in the composition may be, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the composition.

The concentration of mushroom extract in the composition may be, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the composition.

The immunostimulatory compositions of the present disclosure may contain various concentrations of polysaccharides from the mushroom extract. The concentration of polysaccharides from the mushroom extract in the composition may be, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the composition. In some embodiments, the concentration of polysaccharides from the mushroom extract in the composition is at least about 5% of the total dry weight of the composition.

The concentration of polysaccharides from the mushroom extract in the composition may be, for example, about 1% to about 40%, about 10% to about 50%, about 20% to about 60%, about 30% to about 70%, about 40% to about 80%, about 50% to about 90%, or about 60% to about 95% of the total dry weight of the composition. The concentration of polysaccharides from the mushroom extract in the composition may be, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the composition.

The concentration of polysaccharides from the mushroom extract in the composition may be, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the composition.

The contribution of polysaccharides from the mushroom extract to the total dry weight of the polysaccharide content in the immunostimulatory compositions may vary. The concentration of polysaccharides from the mushroom extract in the composition may be, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the polysaccharide content. In some embodiments, the concentration of polysaccharides from the mushroom extract in the composition is about 8% of the total dry weight of the polysaccharide content.

The concentration of polysaccharides from the mushroom extract in the composition may be, for example, about 1% to about 40%, about 10% to about 50%, about 20% to about 60%, about 30% to about 70%, about 40% to about 80%, about 50% to about 90%, or about 60% to about 95% of the total dry weight of the polysaccharide content. The concentration of polysaccharides from the mushroom extract in the composition may be, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the polysaccharide content.

The concentration of polysaccharides from the mushroom extract in the composition may be, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the polysaccharide content.

Astragalus Root Extract

The immunostimulatory compositions of the present disclosure may contain various concentrations of an Astragalus root extract. The Astragalus root extract may be present in the amount of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the β-glucan preparation, the ginseng extract, the mushroom extract, and the Astragalus root extract. In some embodiments, the Astragalus root extract is present at about 35% of the total dry weight of the β-glucan preparation, the ginseng extract, the mushroom extract, and the Astragalus root extract.

The Astragalus root extract may be present in compositions in the amount of, for example, about 1% to about 40%, about 10% to about 50%, about 20% to about 60%, about 30% to about 70%, about 40% to about 80%, about 50% to about 90%, or about 60% to about 95% of the total dry weight of the β-glucan preparation, the ginseng extract, the mushroom extract, and the Astragalus root extract. The Astragalus root extract may be present in compositions in the amount of, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the β-glucan glucan preparation, the ginseng extract, the mushroom extract, and the Astragalus root extract. In some embodiments, the Astragalus root extract is present at about 10% to about 40% of the total dry weight of the β-glucan preparation, the ginseng extract, the mushroom extract, and the Astragalus root extract.

The Astragalus root extract may be present in compositions in the amount of, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the β-glucan preparation, the ginseng extract, the mushroom extract, and the Astragalus root extract.

The immunostimulatory compositions of the present disclosure may contain various concentrations of Astragalus root extract in relation to the total dry weight of the composition. The concentration of Astragalus root extract in the composition may be, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the composition. In some embodiments, the concentration of Astragalus root extract in the composition is at least about 35% of the total dry weight of the composition.

The concentration of Astragalus root extract in the composition may be, for example, about 1% to about 40%, about 10% to about 50%, about 20% to about 60%, about 30% to about 70%, about 40% to about 80%, about 50% to about 90%, or about 60% to about 95% of the total dry weight of the composition. The concentration of Astragalus root extract in the composition may be, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the composition. In some embodiments, the Astragalus root extract is present at about 10% to about 40% of the total dry weight of the composition.

The concentration of Astragalus root extract in the composition may be, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the composition.

The immunostimulatory compositions of the present disclosure may contain various concentrations of polysaccharides from the Astragalus root extract. The concentration of polysaccharides from the Astragalus root extract in the composition may be, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the composition. In some embodiments, the concentration of polysaccharides from the Astragalus root extract in the composition is at least about 5% of the total dry weight of the composition.

The concentration of polysaccharides from the Astragalus root extract in the composition may be, for example, about 1% to about 40%, about 10% to about 50%, about 20% to about 60%, about 30% to about 70%, about 40% to about 80%, about 50% to about 90%, or about 60% to about 95% of the total dry weight of the composition. The concentration of polysaccharides from the Astragalus root extract in the composition may be, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the composition.

The concentration of polysaccharides from the Astragalus root extract in the composition may be, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the composition.

The contribution of polysaccharides from the Astragalus root extract to the total dry weight of the polysaccharide content in the immunostimulatory compositions may vary. The concentration of polysaccharides from the Astragalus root extract in the composition may be, for example, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the total dry weight of the polysaccharide content. In some embodiments, the concentration of polysaccharides from the Astragalus root extract in the composition is about 8% of the total dry weight of the polysaccharide content.

The concentration of polysaccharides from the Astragalus root extract in the composition may be, for example, about 1% to about 40%, about 10% to about 50%, about 20% to about 60%, about 30% to about 70%, about 40% to about 80%, about 50% to about 90%, or about 60% to about 95% of the total dry weight of the polysaccharide content. The concentration of polysaccharides from the Astragalus root extract in the composition may be, for example, about 1% to about 20%, about 10% to about 30%, about 20% to about 40%, about 30% to about 50%, about 40% to about 60%, about 50% to about 70%, about 60% to about 80%, about 70% to about 90%, or about 80% to about 95% of the total dry weight of the polysaccharide content.

The concentration of polysaccharides from the Astragalus root extract in the composition may be, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% of the total dry weight of the polysaccharide content.

Preparing Immunostimulatory Compositions

The present disclosure further provides methods of preparing the immunostimulatory compositions of the present disclosure containing a β-glucan preparation, a ginseng extract, and a mushroom extract. In some embodiments, the immunostimulatory compositions of the present disclosure further contain an Astragalus root extract in addition to a β-glucan preparation, a ginseng extract, and a mushroom extract.

Methods of preparing immunostimulatory compositions are well-known in the art and are described herein. Once an appropriate β-glucan preparation, a ginseng extract, a mushroom extract, and/or an Astragalus root extract have been prepared, these components may be combined into a composition. In some embodiments, one or more of a β-glucan preparation, a ginseng extract, a mushroom extract, and/or an Astragalus root extract have been prepared into an aqueous stock solution. Specific quantities of each component, whether in the form of an aqueous solution, a powder, or other suitable form, may be combined into a composition. The quantities should be selected based on the desired end concentration of each component in the composition.

In an exemplary embodiment, the composition is formulated such that the β-glucan preparation is present in the range of about 40% to about 80% of the total dry weight of the β-glucan glucan preparation, the ginseng extract, and the mushroom extract; the ginseng extract is present in the range of about 10% to about 30% of the total dry weight of the β-glucan preparation, the ginseng extract, and the mushroom extract, and; the mushroom extract is present in the range of about 1% to about 20% of the total dry weight of the β-glucan preparation, the ginseng extract, and the mushroom extract. The percentage ranges described above reflect the concentration of each component with respect to the total dry weight of the β-glucan preparation, the ginseng extract, and the mushroom extract in the composition, and not necessarily the total dry weight of the composition as a whole.

In another exemplary embodiment, the composition is formulated such that the β-glucan glucan preparation is present in the range of about 40% to about 80% of the total dry weight of the β-glucan preparation, the ginseng extract, the mushroom extract, and the Astragalus root extract; the ginseng extract is present in the range of about 10% to about 30% of the total dry weight of the β-glucan preparation, the ginseng extract, the mushroom extract, and the Astragalus root extract, the mushroom extract is present in the range of about 1% to about 20% of the total dry weight of the β-glucan preparation, the ginseng extract, the mushroom extract, and the Astragalus root extract, and; the Astragalus root extract is present in the range of about 10% to about 40% of the total dry weight of the β-glucan preparation, the ginseng extract, the mushroom extract, and the Astragalus root extract. The percentage ranges described above reflect the concentration of each component with respect to the total dry weight of the β-glucan preparation, the ginseng extract, the mushroom extract, and Astragalus root extract in the composition, and not necessarily the total dry weight of the composition as a whole.

In an exemplary embodiment, the composition is formulated such that the β-glucan preparation is present at about 65% to about 70% of the total dry weight of the β-glucan preparation, the ginseng extract, and the mushroom extract; the ginseng extract is from Chinese ginseng and is present at about 20% to about 25% of the total dry weight of the β-glucan preparation, the ginseng extract, and the mushroom extract, and; the mushroom extract is from princess Matsutake mushroom and is present at about 5% to about 10% of the total dry weight of the β-glucan preparation, the ginseng extract, and the mushroom extract.

In an exemplary embodiment, the composition is formulated such that the β-glucan preparation is present at about 75% to about 80% of the total dry weight of the β-glucan preparation, the ginseng extract, and the mushroom extract; the ginseng extract is from Chinese ginseng and is present at about 15% to about 20% of the total dry weight of the β-glucan preparation, the ginseng extract, and the mushroom extract, and; the mushroom extract is from princess Matsutake mushroom and is present at about 1% to about 5% of the total dry weight of the β-glucan preparation, the ginseng extract, and the mushroom extract.

In an exemplary embodiment, the composition is formulated such that the β-glucan preparation is present at about 65% to about 70% of the total dry weight of the β-glucan preparation, the ginseng extract, and the mushroom extract; the ginseng extract is from Chinese ginseng and is present at about 10% to about 15% of the total dry weight of the β-glucan preparation, the ginseng extract, and the mushroom extract, and; the mushroom extract is from princess Matsutake mushroom and is present at about 15% to about 20% of the total dry weight of the β-glucan preparation, the ginseng extract, and the mushroom extract.

In an exemplary embodiment, the composition is formulated such that the β-glucan preparation is present at about 65% to about 70% of the total dry weight of the β-glucan preparation, the ginseng extract, and the mushroom extract; the ginseng extract is from American ginseng and is present at about 20% to about 25% of the total dry weight of the β-glucan preparation, the ginseng extract, and the mushroom extract, and; the mushroom extract is from princess Matsutake mushroom and is present at about 5% to about 10% of the total dry weight of the β-glucan preparation, the ginseng extract, and the mushroom extract.

In an exemplary embodiment, the composition is formulated such that the β-glucan preparation is present at about 65% to about 70% of the total dry weight of the β-glucan preparation, the ginseng extract, and the mushroom extract; the ginseng extract is from Chinese ginseng and is present at about 20% to about 25% of the total dry weight of the β-glucan preparation, the ginseng extract, and the mushroom extract, and; the mushroom extract is from Reishi mushroom and is present at about 5% to about 10% of the total dry weight of the β-glucan preparation, the ginseng extract, and the mushroom extract.

In some embodiments, the immunostimulatory compositions of the present disclosure do not contain both a ginseng extract from American ginseng and a mushroom extract from Reishi mushroom. In these embodiments, the ginseng extract is from a ginseng source other than American ginseng and the mushroom extract is from a mushroom source other than Reishi mushroom. For example, a composition of the present disclosure may not contain both a ginseng extract from American ginseng that is present at about 20% to about 25% of the total dry weight of the β-glucan preparation, the ginseng extract, and the mushroom extract, and a mushroom extract from Reishi mushroom that is present at about 5% to about 10% of the total dry weight of the β-glucan preparation, the ginseng extract, and the mushroom extract.

An exemplary composition containing both a ginseng extract from American ginseng and a mushroom extract from Reishi mushroom that would not be used in the methods and compositions of the present disclosure includes a composition formulated such that the β-glucan preparation is present at about 65% to about 70% of the total dry weight of the β-glucan preparation, the ginseng extract, and the mushroom extract; the ginseng extract is from American ginseng and is present at about 20% to about 25% of the total dry weight of the β-glucan preparation, the ginseng extract, and the mushroom extract, and; the mushroom extract is from Reishi mushroom and is present at about 5% to about 10% of the total dry weight of the β-glucan preparation, the ginseng extract, and the mushroom extract.

In some embodiments, the immunostimulatory compositions of the present disclosure do not contain an Astragalus extract.

The immunostimulatory compositions of the present disclosure may have varying concentrations of total polysaccharide content. Immunostimulatory compositions may contain, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% polysaccharides by total dry weight of the composition.

### Orally Consumable Products

The compositions of the present disclosure may be formulated into an orally consumable product. As used herein, the term “orally consumable product(s)” refers to edible substances which are contacted with the mouth of man or animal, including substances that are taken into and subsequently ejected from the mouth and substances which are drunk, eaten, swallowed, or otherwise ingested; and that are safe for human or animal consumption when used in a generally acceptable range of concentrations. The orally consumable products containing immunostimulatory compositions may be administered to a subject to enhance immune function in the subject. In some embodiments, an immunostimulatory composition formulated into an orally consumable product contains one or more non-immunostimulatory excipients.

Orally consumable products of the present disclosure may take various forms. An orally consumable product of the present disclosure may include, for example, a foodstuff composition, a beverage product, a dietary supplement, a nutraceutical, an edible gel mix, an edible gel composition, and a pharmaceutical composition.

Orally consumable products may also be formulated into a granulated powder, a soft gel composition, and a flash dissolve composition.

Orally consumable products may contain one or more sweeteners. The one or more sweeteners may already be present in the orally consumable product or may be added to the orally consumable product, or one or more compounds or ingredients used to make the orally consumable product. In certain embodiments, the one or more sweeteners may include, for example, natural sweeteners, and artificial or synthetic sweeteners. Suitable sweeteners and combinations of sweeteners may be selected for the desired nutritional characteristics, taste profile, mouthfeel, and other organoleptic factors. In some embodiments, the one or more sweeteners include high intensity sweeteners and/or natural high intensity sweeteners, including, for example, stevia extracts, steviol glycosides, steviosides, rebaudioside A, rebaudioside B, rebaudioside C, rebaudioside D, rebaudioside F, dulcoside A, rubusosides, steviolbiosides, sucrose, high fructose corn syrup, fructose, fructooligosaccharides, glucose, xylose, arabinose, rhamnose, erythritol, xylitol, mannitol, sorbitol, inositol, AceK, aspartame, neotame, oligofructose, sucralose, saccharine, naringin dihydrochalcone (NarDHC), neohesperidin dihydrochalcone (NDHC), rubusoside, mogroside IV, siamenoside I, mogroside V, monatin, thaumatin, monellin, brazzein, L-alanine, glycine, Lo Han Guo, hernandulcin, phyllodulcin, trilobtain, and combinations thereof.

Orally consumable products of the present disclosure may contain one or more additives. The one or more additives may be present to add or enhance one or more characteristics of the orally consumable product, such as flavor, texture, aroma, color, shelf-life, etc. The one or more additives may already be present in the orally consumable product or may be added to the orally consumable product, or one or more compounds or ingredients used to make the orally consumable product. The orally consumable product may contain any suitable additive known in the art. Examples of suitable additives include, for example, carbohydrates, polyols, amino acids or salts thereof, poly-amino acids or salt thereof, sugar acids or salts thereof, nucleotides, organic acids, inorganic acids, organic salts, organic acid salts, organic base salts, inorganic salts, bitter compounds, flavorants, flavoring ingredients, astringent compounds, proteins, protein hydrolysates, surfactants, emulsifiers, flavonoids, alcohols, polymers, preservatives, thickening agents, food colorings, and combinations thereof.

Foodstuff Compositions

In certain embodiments, immunostimulatory compositions of the present disclosure may be present in foodstuff compositions. As used herein, “foodstuff composition(s)” refers to any solid or liquid ingestible material that may, but need not, have a nutritional value and is intended for consumption by man or animal.

Examples of suitable foodstuff compositions may include, for example, beverages (both carbonated and non-carbonated), such as coffee, teas, herbal teas, fruit drinks, soft drinks (e.g., colas), and the like; confectionary compositions, such as candies, mints, fruit flavored drops, cocoa products, chocolates, and the like; condiments, such as ketchup, mustard, mayonnaise, and the like; chewing gums; cereal compositions; baked goods, such as breads, cakes, pies, cookies, and the like; dairy products, such as milk, cheese, cream, ice cream, sour cream, yoghurt, sherbet, and the like; tabletop sweetener compositions; soups; stews; convenience foods; meats, such as ham, bacon, sausages, jerky, and the like; gelatins and gelatin-like products such as jams, jellies, preserves, and the like; fruits; vegetables; egg products; icings; syrups including molasses; snacks; nut meats and nut products; and animal feed.

Foodstuff compositions may also include herbs, spices and seasonings, natural and synthetic flavors, and flavor enhancers. In some embodiments, foodstuff compositions include, for example, prepared packaged products, such as dietetic sweeteners, liquid sweeteners, granulated flavor mixes which upon reconstitution with water provide non-carbonated drinks, instant pudding mixes, instant coffee and tea, coffee whiteners, malted milk mixes, pet foods, livestock feed, and materials for baking applications, such as powdered baking mixes for the preparation of breads, cookies, cakes, pancakes, donuts and the like.

Beverage Products

In certain embodiments, immunostimulatory compositions of the present disclosure may be present in beverage products. Beverage products of the present disclosure include both carbonated and non-carbonated beverage products. Examples of suitable beverage products include, for example, soft drinks, fountain beverages, frozen beverages, ready-to-drink beverages, coffee, teas, dairy beverages, powdered soft drinks, liquid concentrates, flavored water, enhanced water, fruit juices, fruit juice flavored drinks, sport drinks, energy drinks, and alcoholic beverages, such as beers, wines, and liquors.

In some embodiments, a beverage product of the present disclosure includes one or more beverage ingredients including, for example, acidulants, fruit juices and/or vegetable juices, pulp, etc., flavorings, coloring, preservatives, vitamins, minerals, electrolytes, erythritol, tagatose, glycerine, and carbon dioxide. Such beverage products may be provided in any suitable form, such as a beverage concentrate or a carbonated, ready-to-drink beverage.

In certain embodiments, beverage products of the present disclosure may have any of numerous different specific formulations or constitutions. The formulation of a beverage product of the present disclosure may vary to a certain extent, depending upon such factors as the product's intended market segment, its desired nutritional characteristics, flavor profile, and the like. For example, in certain embodiments, it will generally be an option to add further ingredients to the formulation of a particular beverage product. For example, sweeteners, flavorings, electrolytes, vitamins, fruit juices or other fruit products, tastents, masking agents and the like, flavor enhancers, and/or carbonation typically may be added to any such formulations to vary the taste, mouthfeel, nutritional characteristics, etc. In some embodiments, orally consumable products are formulated to exhibit a particular flavor(s). Flavors that may be used may include, for example, vanilla flavor, chocolate flavor, banana flavor, strawberry flavor, and various others that will be readily apparent to one of skill in the art. Exemplary additional flavorings include, for example, cola flavoring, citrus flavoring, and spice flavorings. In some embodiments, carbonation in the form of carbon dioxide may be added for effervescence. In other embodiments, preservatives may be added, depending upon the other ingredients, production technique, desired shelf life, etc. In certain embodiments, caffeine may be added. In some embodiments, the beverage product is a cola-flavored carbonated beverage, characteristically containing carbonated water, sweetener, kola nut extract and/or other flavoring, caramel coloring, one or more acids, and optionally other ingredients.

Dietary Supplements and Nutraceuticals

In certain embodiments, immunostimulatory compositions of the present disclosure may be present in dietary supplements. As used herein, “dietary supplement(s)” refers to compounds intended to supplement the diet and provide nutrients, such as vitamins, minerals, fiber, fatty acids, amino acids, etc. that may be missing or may not be consumed in sufficient quantities in a diet. Any suitable dietary supplement known in the art may be used. Examples of suitable dietary supplements include, for example, nutrients, vitamins, minerals, fiber, fatty acids, herbs, botanicals, amino acids, and metabolites.

In some embodiments, immunostimulatory compositions of the present disclosure are present in nutraceuticals. As used herein, “nutraceutical(s)” refers to compounds, which includes any food or part of a food that may provide medicinal or health benefits, including the prevention and/or treatment of disease or disorder (e.g., fatigue, insomnia, effects of aging, memory loss, mood disorders, cardiovascular disease and high levels of cholesterol in the blood, diabetes, osteoporosis, inflammation, autoimmune disorders, etc.). Any suitable nutraceutical known in the art may be used. In some embodiments, nutraceuticals can be used as supplements to food and beverages and as pharmaceutical formulations for enteral or parenteral applications which may be solid formulations, such as capsules or tablets, or liquid formulations, such as solutions or suspensions.

In some embodiments, dietary supplements and nutraceuticals may further contain protective hydrocolloids (such as gums, proteins, modified starches), binders, film-forming agents, encapsulating agents/materials, wall/shell materials, matrix compounds, coatings, emulsifiers, surface active agents, solubilizing agents (oils, fats, waxes, lecithins, etc.), adsorbents, carriers, fillers, co-compounds, dispersing agents, wetting agents, processing aids (solvents), flowing agents, taste-masking agents, weighting agents, jellyfying agents, gel-forming agents, antioxidants and antimicrobials.

Edible Gel Mixes and Gel Compositions

In certain embodiments, immunostimulatory compositions of the present disclosure may be present in gel mixes and gel compositions. As used herein, a “gel” refers to a colloidal system in which a network of particles spans the volume of a liquid medium. Although gels mainly are composed of liquids, and thus exhibit densities similar to liquids, gels have the structural coherence of solids due to the network of particles that spans the liquid medium. For this reason, gels generally appear to be solid, jelly-like materials. Gels that can be eaten are referred to as “edible gel compositions.” Edible gel compositions typically are eaten as snacks, as desserts, as a part of staple foods, or along with staple foods. Examples of suitable edible gel compositions include, for example, gel desserts, puddings, jams, jellies, pastes, trifles, aspics, marshmallows, gummy candies, and the like. In some embodiments, edible gel mixes generally are powdered or granular solids to which a fluid may be added to form an edible gel composition. Examples of suitable fluids include, for example, water, dairy fluids, dairy analogue fluids, juices, alcohol, alcoholic beverages, and combinations thereof. Examples of suitable dairy fluids include, for example, milk, cultured milk, cream, fluid whey, and mixtures thereof. Examples of suitable dairy analogue fluids include, for example, soy milk and non-dairy coffee whitener.

As used herein, the term “gelling ingredient” refers to any material that can form a colloidal system within a liquid medium. Examples of suitable gelling ingredients include, for example, gelatin, alginate, carageenan, gum, pectin, konjac, agar, food acid, rennet, starch, starch derivatives, and combinations thereof. It is well known to those having ordinary skill in the art that the amount of gelling ingredient used in an edible gel mix or an edible gel composition varies considerably depending on a number of factors including, for example, the particular gelling ingredient used, the particular fluid base used, and the desired properties of the gel.

Gel mixes and gel compositions of the present disclosure may be prepared by any suitable method known in the art. In some embodiments, edible gel mixes and edible gel compositions of the present disclosure may be prepared using other ingredients in addition to an immunostimulatory composition of the present disclosure and the gelling agent. Examples of other suitable ingredients include, for example, a food acid, a salt of a food acid, a buffering system, a bulking agent, a sequestrant, a cross-linking agent, one or more flavors, one or more colors, and combinations thereof.

Pharmaceutical Compositions

In certain embodiments, immunostimulatory compositions of the present disclosure may be present in pharmaceutical compositions. Any suitable pharmaceutical composition known in the art may be used. In certain embodiments, a pharmaceutical composition of the present disclosure contains an immunostimulatory composition of the present disclosure and one or more pharmaceutically acceptable excipients. In some embodiments, pharmaceutical compositions of the present disclosure may be used to formulate pharmaceutical drugs containing one or more active agents that exert a biological effect. Accordingly, in some embodiments, pharmaceutical compositions of the present disclosure may contain one or more active agents that exert a biological effect. Suitable active agents are well known in the art (e.g., The Physician's Desk Reference). Such compositions can be prepared according to procedures well known in the art, for example, as described in Remington's Pharmaceutical Sciences, Mack Publishing Co., Easton, Pa., USA.

Examples of suitable active agents include, for example, bronchodilators, anorexiants, antihistamines, nutritional supplements, laxatives, analgesics, anesthetics, antacids, H.sub.2-receptor antagonists, anticholinergics, antidiarrheals, demulcents, antitussives, antinauseants, antimicrobials, antibacterials, antifungals, antivirals, expectorants, anti-inflammatory agents, antipyretics, and mixtures thereof. In one embodiment, the active agent is selected from the group consisting of antipyretics and analgesics, e.g., ibuprofen, acetaminophen, or aspirin; laxatives, e.g., phenolphthalein dioctyl sodium sulfosuccinate; appetite depressants, e.g., amphetamines, phenylpropanolamine, phenylpropanolamine hydrochloride, or caffeine; antacidics, e.g., calcium carbonate; antiasthmatics, e.g., theophylline; antidiuretics, e.g., diphenoxylate hydrochloride; agents active against flatulence, e.g., simethecon; migraine agents, e.g., ergotaminetartrate; psychopharmacological agents, e.g., haloperidol; spasmolytics or sedatives, e.g., phenobarbitol; antihyperkinetics, e.g., methyldopa or methylphenidate; tranquilizers, e.g., benzodiazepines, hydroxinmeprobramates or phenothiazines; antihistaminics, e.g., astemizol, chloropheniramine maleate, pyridamine maleate, doxlamine succinate, bromopheniramine maleate, phenyltoloxamine citrate, chlorocyclizine hydrochloride, pheniramine maleate, and phenindamine tartrate; decongestants, e.g., phenylpropanolamine hydrochloride, phenylephrine hydrochloride, pseudoephedrine hydrochloride, pseudoephedrine sulfate, phenylpropanolamine bitartrate, and ephedrine; beta-receptor blockers, e.g., propanolol; agents for alcohol withdrawal, e.g., disulfiram; antitussives, e.g., benzocaine, dextromethorphan, dextromethorphan hydrobromide, noscapine, carbetapentane citrate, and chlophedianol hydrochloride; fluorine supplements, e.g., sodium fluoride; local antibiotics, e.g., tetracycline or cleocine; corticosteroid supplements, e.g., prednisone or prednisolone; agents against goiter formation, e.g., colchicine or allopurinol; antiepileptics, e.g., phenytoine sodium; agents against dehydration, e.g., electrolyte supplements; antiseptics, e.g., cetylpyridinium chloride; NSAIDs, e.g., acetaminophen, ibuprofen, naproxen, or salts thereof; gastrointestinal active agents, e.g., loperamide and famotidine; various alkaloids, e.g., codeine phosphate, codeine sulfate, or morphine; supplements for trace elements, e.g., sodium chloride, zinc chloride, calcium carbonate, magnesium oxide, and other alkali metal salts and alkali earth metal salts; vitamins; ion-exchange resins, e.g., cholestyramine; cholesterol-depressant and lipid-lowering substances; antiarrhythmics, e.g., N-acetylprocainamide; and expectorants, antibacterial agents such as ciprofloxacin, ofloxacin, and pefloxacin; antiepileptics such as zonisamide; macrolide antibiotics such as erythromycin; beta-lactam antibiotics such as penicillins and cephalosporins; psychotropic active substances such as chlorpromazine; active substances such as sulpyrine; and agents active against ulcers, such as cimetidine. In some embodiments, the pharmaceutical composition of the present invention contains at least one amino acid selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

In some embodiments, a pharmaceutical composition of the present disclosure is a liquid dosage form for oral administration including, for example, pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (such as cottonseed, groundnut, corn, germ, olive, castor, and sesame oils), glycerol, tetrahydrofurfuryl alcohol, polyethylene glycols and fatty acid esters of sorbitan, and mixtures thereof. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

In other embodiments, pharmaceutical compositions of the present disclosure can be in the form of chewable tablets (e.g., U.S. Pat. Nos. 4,684,534 and 6,060,078); orally disintegrating compositions (e.g., U.S. Pat. Nos. 6,368,625 and 6,316,029); solid dosage forms, such as water and/or saliva activated effervescent granule (e.g., U.S. Pat. No. 6,649,186); and film-shaped or wafer-shaped pharmaceutical compositions; gum base formulations having a medicament or agent and an immunostimulatory composition of the present disclosure contained in a coating that surrounds the gum base formulation (e.g., U.S. Pat. No. 6,773,716).

### Enhancing Immune Function

The present disclosure provides methods of enhancing immune function in a subject. Methods of enhancing immune function in a subject may involve administering to the subject an immunostimulatory composition of the present disclosure.

In some embodiments, a subject is administered an immunostimulatory composition containing a β-glucan preparation, a ginseng extract, and a mushroom extract, where the composition is formulated such that the β-glucan preparation is present in the range of about 40% to about 80% of the total dry weight of the β-glucan preparation, the ginseng extract, and the mushroom extract; the ginseng extract is present in the range of about 10% to about 30% of the total dry weight of the β-glucan preparation, the ginseng extract, and the mushroom extract, and; the mushroom extract is present in the range of about 1% to about 20% of the total dry weight of the β-glucan preparation, the ginseng extract, and the mushroom extract. In some embodiments, the immunostimulatory composition containing a β-glucan preparation, a ginseng extract, and a mushroom extract is formulated into an orally consumable product for administration to a subject.

In some embodiments, a subject is administered an immunostimulatory composition containing a β-glucan preparation, a ginseng extract, a mushroom extract, and an Astragalus root extract, where the composition is formulated such that the β-glucan preparation is present in the range of about 40% to about 80% of the total dry weight of the β-glucan preparation, the ginseng extract, the mushroom extract, and the Astragalus root extract; the ginseng extract is present in the range of about 10% to about 30% of the total dry weight of the β-glucan preparation, the ginseng extract, the mushroom extract, and the Astragalus root extract, the mushroom extract is present in the range of about 1% to about 20% of the total dry weight of the β-glucan preparation, the ginseng extract, the mushroom extract, and the Astragalus root extract, and; the Astragalus root extract is present in the range of about 10% to about 40% of the total dry weight of the β-glucan preparation, the ginseng extract, the mushroom extract, and the Astragalus root extract. In some embodiments, the immunostimulatory composition containing a β-glucan preparation, a ginseng extract, a mushroom extract, and an Astragalus root extract is formulated into an orally consumable product for administration to a subject.

In an exemplary embodiment, a subject is administered an immunostimulatory composition containing a β-glucan preparation, a ginseng extract, and a mushroom extract, where the composition is formulated such that the β-glucan preparation is present at about 65% to about 70% of the total dry weight of the β-glucan preparation, the ginseng extract, and the mushroom extract; the ginseng extract is from Chinese ginseng and is present at about 20% to about 25% of the total dry weight of the β-glucan preparation, the ginseng extract, and the mushroom extract, and; the mushroom extract is from princess Matsutake mushroom and is present at about 5% to about 10% of the total dry weight of the β-glucan preparation, the ginseng extract, and the mushroom extract. This composition may be formulated into an orally consumable product for administration to a subject.

In an exemplary embodiment, a subject is administered an immunostimulatory composition containing a β-glucan preparation, a ginseng extract, and a mushroom extract, where the composition is formulated such that the β-glucan preparation is present at about 75% to about 80% of the total dry weight of the β-glucan preparation, the ginseng extract, and the mushroom extract; the ginseng extract is from Chinese ginseng and is present at about 15% to about 20% of the total dry weight of the β-glucan preparation, the ginseng extract, and the mushroom extract, and; the mushroom extract is from princess Matsutake mushroom and is present at about 1% to about 5% of the total dry weight of the β-glucan preparation, the ginseng extract, and the mushroom extract. This composition may be formulated into an orally consumable product for administration to a subject.

In an exemplary embodiment, a subject is administered an immunostimulatory composition containing a β-glucan preparation, a ginseng extract, and a mushroom extract, where the composition is formulated such that the β-glucan preparation is present at about 65% to about 70% of the total dry weight of the β-glucan preparation, the ginseng extract, and the mushroom extract; the ginseng extract is from Chinese ginseng and is present at about 10% to about 15% of the total dry weight of the β-glucan preparation, the ginseng extract, and the mushroom extract, and; the mushroom extract is from princess Matsutake mushroom and is present at about 15% to about 20% of the total dry weight of the β-glucan preparation, the ginseng extract, and the mushroom extract. This composition may be formulated into an orally consumable product for administration to a subject.

In an exemplary embodiment, a subject is administered an immunostimulatory composition containing a β-glucan preparation, a ginseng extract, and a mushroom extract, where the composition is formulated such that the β-glucan preparation is present at about 65% to about 70% of the total dry weight of the β-glucan preparation, the ginseng extract, and the mushroom extract; the ginseng extract is from American ginseng and is present at about 20% to about 25% of the total dry weight of the β-glucan preparation, the ginseng extract, and the mushroom extract, and; the mushroom extract is from princess Matsutake mushroom and is present at about 5% to about 10% of the total dry weight of the β-glucan preparation, the ginseng extract, and the mushroom extract. This composition may be formulated into an orally consumable product for administration to a subject.

In an exemplary embodiment, a subject is administered an immunostimulatory composition containing a β-glucan preparation, a ginseng extract, and a mushroom extract, where the composition is formulated such that the β-glucan preparation is present at about 65% to about 70% of the total dry weight of the β-glucan preparation, the ginseng extract, and the mushroom extract; the ginseng extract is from Chinese ginseng and is present at about 20% to about 25% of the total dry weight of the β-glucan preparation, the ginseng extract, and the mushroom extract, and; the mushroom extract is from Reishi mushroom and is present at about 5% to about 10% of the total dry weight of the β-glucan preparation, the ginseng extract, and the mushroom extract. This composition may be formulated into an orally consumable product for administration to a subject.

In some embodiments, an immunostimulatory composition to be administered to a subject of the present disclosure does not contain both a ginseng extract from American ginseng and a mushroom extract from Reishi mushroom. In these embodiments, the ginseng extract is from a ginseng source other than American ginseng and the mushroom extract is from a mushroom source other than Reishi mushroom. For example, a composition of the present disclosure may not contain both a ginseng extract from American ginseng that is present at about 20% to about 25% of the total dry weight of the β-glucan preparation, the ginseng extract, and the mushroom extract, and a mushroom extract from Reishi mushroom that is present at about 5% to about 10% of the total dry weight of the β-glucan preparation, the ginseng extract, and the mushroom extract.

In some embodiments, an immunostimulatory composition to be administered to a subject of the present disclosure does not contain an Astragalus extract.

Subjects

In some embodiments, immunostimulatory compositions of the present disclosure can be administered to a subject in any form suitable to achieve their intended purpose. In certain embodiments, the composition is one which can be administered orally. Suitable subjects include, for example, any animal with an immune system, such as a human. Other suitable subjects include, for example, canines, felines, dogs, cats, hamsters, gerbils, livestock, horses, cattle, sheep, fish, and the like. A veterinary composition, as used herein, refers to a pharmaceutical composition that is suitable for non-human animals. Such veterinary compositions are well known in the art.

Immune Function

Various assays to probe enhanced immune function in a subject are known in the art and are described herein. For example, enhanced immune function may manifest as enhanced in vivo delayed-type hypersensitivity (including contact hypersensitivity) responses against antigens to which the subject was previously exposed to, sensitized to, or vaccinated against. Enhanced immune function in a subject may also involve enhanced cellular proliferation, cytokine production, oxidative burst response, or phagocytic activity in response to pathogen-related, pathogen-mimicking, mitogenic stimuli, or antigens which the subject was previously exposed to, sensitized to, or vaccinated against.

Investigating the status of immune function in a subject may also involve, for example, measuring antibody levels and/or levels of one or more other markers (e.g. complement protein production) in blood serum or other bodily samples obtained from the subject. An increase in antibody production in response to pathogenic stimuli or vaccination is a sign of improved humoral immunity. Enhanced immune function may include enhanced lymphocyte proliferation and/or enhanced production of one or more cytokines in a subject. In some embodiments, the cytokines having enhanced production in a subject include IFN-γ, IL-12, and IL-10.

Immunostimulatory compositions of the present disclosure may be used to enhance immune function in a subject such that the ability of a subject to respond to an acute pathogenic stimulus is enhanced. For example, immunostimulatory compositions of the present disclosure may be used for enhancing protective immune responses to an exogenous pathogen or to an autologous trigger, such as neoplastic cells. In this sense, the immunostimulatory compositions disclosed herein may be used as a prophylactic, e.g. to avoid an infection or reduce the severity of an infection or another immune-related disease.

Immunostimulatory compositions of the present disclosure may enhance immune function in a subject by, for example, increasing the activity or number of natural killer (NK) cells, increasing pathogen-specific or tumor-specific IgA, IgG or IgM antibody production, increasing IgA, IgG or IgM concentrations to normal range values in the blood of mammals with sub-normal levels of said antibodies, increasing the total number of white blood cells in mammals with treatment- or disease-induced decreased number of white blood cells, increasing the number or activity of phagocytic cells in mammals with treatment- or disease-induced decreased number of phagocytic cells, improving cell-mediated cytotoxic responses against pathogens, pathogen-infected cells or tumour cells, improving the barrier function of mucosal epithelia by increasing slgA excretion, increasing intestinal mucus production, decreasing epithelial permeability or decreasing microbial translocation across the epithelia with the exception of non-infectious uptake of microbes by local immune cells, improving the composition of the intestinal microbiota by increasing the number or activity of beneficial bacteria such as bifidobacteria or lactobacilli, by improving the colonization resistance of the intestinal microbiota, by decreasing the number of potential pathogenic organisms in the intestinal microbiota, or by decreasing the pH of the intestinal content.

Methods for enhancing immune function in a subject by administering to the subject an immunostimulatory composition of the present disclosure may involve treating any reduced immune function in a subject caused by, or resulting from, aging, disease, disorder, and/or trauma, for example. In some embodiments, reduced immune function may manifest as a symptom of, for example, infections, inflammations, vascular complications, bad wound healing, mucositis and stomatitis. Involuntary weight loss and sarcopenia may further deteriorate the immune function of a subject.

An immunostimulatory composition of the present disclosure may be used alone or in combination with other medications for treating a subject suffering from a disease or disorder including, for example, cancer, tumor growth, diabetes, chronic obstructive pulmonary disease, bacterial diseases, viral diseases such as HIV-infections, renal disease, renal failure, autoimmune diseases, and heart failure. In an embodiment where an immunostimulatory composition of the present disclosure is used to treat trauma, the trauma may include, for example, surgery, drug treatment, chemotherapy, and radiotherapy.

Administering Immunostimulatory Compositions

The methods of the present disclosure may involve administering an effective amount of an immunostimulatory composition of the present disclosure to a subject. The term “effective amount” as used herein refers to an amount of a compound or composition sufficient to enhance an immune response and ameliorate, palliate, lessen, and/or delay one or more symptoms of a disorder, condition, or disease. An effective amount of the immunostimulatory composition may or may not be achieved in conjunction with another drug, pharmaceutical composition, or treatment. Thus, an “effective amount” may be considered in the context of administering one or more therapeutic agents, and a single agent may be considered to be given in an effective amount if, in conjunction with one or more other agents, a desirable result may be or is achieved.

To enhance immune function in a subject according to the methods of the present disclosure, various dosage forms of components of the immunostimulatory compositions of the present disclosure may be used. The doses in the following paragraphs may be administered to a subject such as, for example, a human.

With respect to the quantity of β-glucan from a β-glucan preparation in an immunostimulatory composition of the present disclosure, the quantity of β-glucan administered may be, for example, about 20 mg to about 1,000 mg, about 20 mg to about 500 mg, about 20 mg to about 200 mg, about 100 mg to about 500 mg, about 100 mg to about 300 mg, about 100 mg to about 200 mg, about 150 mg to about 500 mg, about 150 mg to about 300 mg, about 150 mg to about 250 mg, or about 150 mg to about 200 mg. With respect to the quantity of β-glucan from a β-glucan preparation in an immunostimulatory composition of the present disclosure, the quantity of β-glucan administered may be, for example, at least about 50 mg, at least about 75 mg, at least about 100 mg, at least about 125 mg, at least about 150 mg, at least about 175 mg, at least about 200 mg, at least about 225 mg, at least about 250 mg, at least about 275 mg, at least about 300 mg, at least about 400 mg, or at least about 500 mg. In some embodiments, with respect to the quantity of β-glucan from a β-glucan preparation in an immunostimulatory composition of the present disclosure, the quantity of β-glucan administered is about 100 mg to about 300 mg.

With respect to the quantity of polysaccharides from a ginseng extract in an immunostimulatory composition of the present disclosure, the quantity of polysaccharides from the ginseng extract administered may be, for example, about 5 mg to about 300 mg, about 6 mg to about 300 mg, about 7 mg to about 300 mg, about 8 mg to about 300 mg, about 9 mg to about 300 mg, about 10 mg to about 300 mg, about 5 mg to about 100 mg, about 7.5 mg to about 100 mg, about 5 mg to about 50 mg, or about 7.5 mg to about 50 mg. With respect to the quantity of polysaccharides from a ginseng extract in an immunostimulatory composition of the present disclosure, the quantity of polysaccharides from the ginseng extract administered may be, for example, at least about 5 mg, at least about 6 mg, at least about 7 mg, at least about 8 mg, at least about 9 mg, at least about 10 mg, at least about 15 mg, at least about 30 mg, at least about 40 mg, at least about 50 mg, at least about 60 mg, at least about 75 mg, at least about 100 mg, or at least about 200 mg. In some embodiments, with respect to the quantity of polysaccharides from a ginseng extract in an immunostimulatory composition of the present disclosure, the quantity of polysaccharides from the ginseng extract administered is about 5 mg to about 100 mg.

With respect to the quantity of polysaccharides from a mushroom extract in an immunostimulatory composition of the present disclosure, the quantity of polysaccharides from the mushroom extract administered may be, for example, about 10 mg to about 300 mg, about 10 mg to about 50 mg, about 12 mg to about 300 mg, about 15 mg to about 300 mg, about 25 mg to about 200 mg, about 50 mg to about 200 mg, about 50 mg to about 150 mg, about 50 mg to about 100 mg, about 75 mg to about 100 mg, about 10 mg to about 100 mg, or about 10 mg to about 75 mg. With respect to the quantity of polysaccharides from a mushroom extract in an immunostimulatory composition of the present disclosure, the quantity of polysaccharides from the mushroom extract administered may be, for example, at least about 10 mg, at least about 12 mg, at least about 14 mg, at least about 16 mg, at least about 18 mg, at least about 20 mg, at least about 25 mg, at least about 30 mg, at least about 40 mg, at least about 50 mg, at least about 60 mg, at least about 75 mg, at least about 100 mg, or at least about 200 mg. In some embodiments, with respect to the quantity of polysaccharides from a mushroom extract in an immunostimulatory composition of the present disclosure, the quantity of polysaccharides from the mushroom extract administered is about 10 mg to about 100 mg.

In some embodiments, the immunostimulatory composition administered contains about 100 mg to about 300 mg β-glucan from a β-glucan preparation, about 5 mg to about 100 mg polysaccharides from a ginseng extract, and about 10 mg to about 100 mg polysaccharides from a mushroom extract. In some embodiments, the immunostimulatory composition administered contains at least about 150 mg, about 175 mg, or about 200 mg β-glucan from a β-glucan preparation, at least about 5 mg, about 6 mg, about 7 mg, about 8 mg, about 9 mg, or about 10 mg polysaccharides from a ginseng extract, and at least about 10 mg, at least about 12 mg, at least about 14 mg, or at least about 16 mg polysaccharides from a mushroom extract.

To enhance immune function in a subject, the appropriate dosage of an immunostimulatory composition of the present disclosure (when used alone or in combination with one or more other additional therapeutic agents) may depend on the type of disease to be treated or prevented, the severity and course of the disease or disorder if already present in the subject, whether the immunostimulatory composition is administered for preventive or therapeutic purposes, previous therapy and type of therapy received by the subject, the subject's clinical history, and the discretion of any attending physician if applicable.

The dosage of an immunostimulatory composition of the present disclosure may be administered to a subject at one time or over a series of administrations. For example, the immunostimulatory composition may be administered to a subject, for example, once daily, twice daily, three or more times daily, once every two days, once every three days, once every four days, once every five days, once every six days, once every week, once every two weeks, or once every month or longer. For repeated administrations over several days or longer, depending on the condition, the administration would generally be sustained until a desired suppression of disease and/or disorder symptoms occurs or until there is sufficient confidence that disease and/or disorder symptoms will not appear or that any disease and/or disorder symptoms will have reduced severity upon appearance. The duration of the administration of the immunostimulatory compositions may be, for example, at least one day, at least three days, at least one week, at least two weeks, at least one month, at least two months, at least three months, at least six months, at least nine months, or at least one year or longer.

In some embodiments, the immunostimulatory composition includes about 220 mg to about 250 mg of a β-glucan preparation, about 75 to about 90 mg of a ginseng extract, and about 20 mg to about 40 mg of a mushroom extract. In some embodiments, the composition includes about 220 mg, about 230 mg, about 240 mg, or about 250 mg of a β-glucan preparation. In some embodiments, the composition includes about 80 mg, about 82 mg, about 84 mg, about 86 mg, about 88 mg, or about 90 mg of a ginseng extract. In some embodiments, the composition includes about 20 mg, about 25 mg, about 30 mg, about 33 mg, about 35 mg, about 38 mg, or about 40 mg of a mushroom extract. In some embodiments, the composition includes about 220 mg, about 230 mg, about 240 mg, or about 250 mg of a β-glucan preparation; about 80 mg, about 82 mg, about 84 mg, about 86 mg, about 88 mg, or about 90 mg of a ginseng extract; and about 20 mg, about 25 mg, about 30 mg, about 33 mg, about 35 mg, about 38 mg, or about 40 mg of a mushroom extract. In some embodiments, the compositions described above are administered to a subject once daily. The compositions described above may also be administered to a subject, for example, once every two days, once every three days, once every four days, or once every week.

In some embodiments, the immunostimulatory composition includes about 250 mg to about 325 mg of a β-glucan preparation, about 90 to about 120 mg of a ginseng extract, and about 30 mg to about 50 mg of a mushroom extract. In some embodiments, the composition includes the composition includes about 250 mg, about 265 mg, about 275 mg, about 290 mg, about 300 mg, about 310 mg, or about 325 mg of a β-glucan preparation. In some embodiments, the composition includes about 90 mg, about 95 mg, about 100 mg, about 105 mg, about 110 mg, about 115 mg, or about 120 mg of a ginseng extract. In some embodiments, the composition includes about 30 mg, about 35 mg, about 40 mg, about 45 mg, or about 50 mg of a mushroom extract. In some embodiments, the composition includes about 250 mg, about 265 mg, about 275 mg, about 290 mg, about 300 mg, about 310 mg, or about 325 mg of a β-glucan preparation; about 90 mg, about 95 mg, about 100 mg, about 105 mg, about 110 mg, about 115 mg, or about 120 mg of a ginseng extract of a ginseng extract; and about 30 mg, about 35 mg, about 40 mg, about 45 mg, or about 50 mg of a mushroom extract. In some embodiments, the compositions described above are administered to a subject once daily. The compositions described above may also be administered to a subject, for example, once every two days, once every three days, once every four days, or once every week.

In some embodiments, the immunostimulatory composition includes about 150 mg to about 200 mg β-glucans from a β-glucan preparation, about 5 mg to about 10 mg polysaccharides from a ginseng extract, and about 8 mg to about 16 mg polysaccharides from a mushroom extract. In some embodiments, the composition includes about 150 mg β-glucans, about 175 mg β-glucans, or about 200 mg β-glucans from a β-glucan preparation. In some embodiments, the composition includes about 5 mg, about 6 mg, about 7 mg, about 8 mg, about 9 mg, or about 10 mg polysaccharides from a ginseng extract. In some embodiments, the composition includes about 8 mg, about 9 mg, about 10 mg, about 11 mg, about 12 mg, about 13 mg, about 14 mg, about 15 mg, or about 16 mg polysaccharides from a mushroom extract. In some embodiments, the composition includes about 150 mg β-glucans, about 175 mg β-glucans, or about 200 mg β-glucans from a β-glucan preparation; about 5 mg, about 6 mg, about 7 mg, about 8 mg, about 9 mg, or about 10 mg polysaccharides from a ginseng extract; and about 8 mg, about 9 mg, about 10 mg, about 11 mg, about 12 mg, about 13 mg, about 14 mg, about 15 mg, or about 16 mg polysaccharides from a mushroom extract. In some embodiments, the compositions described above are administered to a subject once daily. The compositions described above may also be administered to a subject, for example, once every two days, once every three days, once every four days, or once every week.

In some embodiments, the immunostimulatory composition includes about 150 mg to about 200 mg β-glucans from a β-glucan preparation, about 20 mg to about 30 mg polysaccharides from a ginseng extract, and about 10 mg to about 20 mg polysaccharides from a mushroom extract. In some embodiments, the composition includes about 150 mg β-glucans, about 175 mg β-glucans, or about 200 mg β-glucans from a β-glucan preparation. In some embodiments, the composition includes about 20 mg, about 25 mg, or about 30 mg polysaccharides from a ginseng extract. In some embodiments, the composition includes about 10 mg, about 12 mg, about 14 mg, about 16 mg, about 18 mg, or about 20 mg polysaccharides from a mushroom extract. In some embodiments, the composition includes about 150 mg β-glucan glucans, about 175 mg β-glucans, or about 200 mg β-glucans from a β-glucan preparation; about 20 mg, about 25 mg, or about 30 mg polysaccharides from a ginseng extract; and about 10 mg, about 12 mg, about 14 mg, about 16 mg, about 18 mg, or about 20 mg polysaccharides from a mushroom extract. In some embodiments, the compositions described above are administered to a subject once daily. The compositions described above may also be administered to a subject, for example, once every two days, once every three days, once every four days, or once every week.

In some embodiments, the immunostimulatory composition includes about 175 mg to about 225 mg β-glucans from a β-glucan preparation, about 25 mg to about 45 mg polysaccharides from a ginseng extract, and about 15 mg to about 30 mg polysaccharides from a mushroom extract. In some embodiments, the composition includes about 175 mg β-glucans, about 195 mg β-glucans, about 200 mg β-glucans, about 205 mg β-glucans, about 210 mg β-glucan glucans, about 215 mg β-glucans, or about 225 mg β-glucans from a β-glucan preparation. In some embodiments, the composition includes about 25 mg, about 28 mg, about 30 mg, about 32 mg, about 35 mg, about 38 mg, about 40 mg, about 42 mg, or about 45 mg polysaccharides from a ginseng extract. In some embodiments, the composition includes about 15 mg, about 18 mg, about 20 mg, about 22 mg, about 24 mg, about 25 mg, or about 30 mg polysaccharides from a mushroom extract. In some embodiments, the composition includes about 175 mg β-glucans, about 195 mg β-glucans, about 200 mg β-glucans, about 205 mg β-glucans, about 210 mg β-glucan glucans, about 215 mg β-glucans, or about 225 mg β-glucans from a β-glucan preparation; about 25 mg, about 28 mg, about 30 mg, about 32 mg, about 35 mg, about 38 mg, about 40 mg, about 42 mg, or about 45 mg polysaccharides from a ginseng extract; and about 15 mg, about 18 mg, about 20 mg, about 22 mg, about 24 mg, about 25 mg, or about 30 mg polysaccharides from a mushroom extract. In some embodiments, the compositions described above are administered to a subject once daily. The compositions described above may also be administered to a subject, for example, once every two days, once every three days, once every four days, or once every week.

In some embodiments related to administration of an immunostimulatory composition of the present disclosure to a subject, the composition is administered such that not more than 250 mg of β-glucans are administered in a single day. In some embodiments related to administration of an immunostimulatory composition of the present disclosure to a subject, the composition is administered such that not more than 3 grams of ginseng extract are administered in a single day.

The progress of administration regimens of immunostimulatory compositions are easily monitored by conventional techniques and assays. In some embodiments, the progress of the administration is monitored by monitoring viral load in a subject. In some embodiments, the progress of the administration is monitored by monitoring the status of disease and/or disorder symptoms in a subject.

Kits

Also provided herein are kits for enhancing immune function in a subject. The kit may contain, for example, an immunostimulatory composition containing β-glucan preparation, a ginseng extract, and a mushroom extract. The kit may contain, for example, an orally consumable product containing an immunostimulatory composition containing a β-glucan preparation, a ginseng extract, and a mushroom extract. The kit may contain, for example, an immunostimulatory composition containing a β-glucan preparation, a ginseng extract, a mushroom extract, and an Astragalus root extract. The kit may contain, for example, an orally consumable product containing an immunostimulatory composition containing a β-glucan preparation, a ginseng extract, a mushroom extract, and an Astragalus root extract.

In some embodiments, the kit contains instructions for administering an immunostimulatory composition of the present disclosure to a subject. In some embodiments, the immunostimulatory composition is formulated into an orally consumable product of the present disclosure. The instructions may provide information related to, for example, suggested guidelines for dosing regimens and duration of administration of the immunostimulatory compositions. The immunostimulatory composition present in the kit may also be provided in a package. Immunostimulatory compositions of the present disclosure may be provided in, for example, a paper package, a plastic package, or one of various other suitable packages well-known to those of skill in the art.

### Examples

The following examples are offered for illustrative purposes and to aid one of skill in better understanding the various embodiments of the disclosure. The following examples are not intended to limit the scope of the present disclosure in any way.

### Example 1: Evaluation of Immune Stimulation Elicited by “Blend A”

The Example demonstrates that the “Blend A” solution (containing β-glucans, ginseng extracts, and mushroom extracts) is a potent immune stimulator. “Blend A” was able to stimulate increased lymphocyte proliferation and increased accumulation of multiple immunostimulation protein markers as compared to a control solution containing β-glucan, despite “Blend A” having a lower total polysaccharide content than the control solution.

### Introduction

β-glucans, a type of polysaccharide consisting of chains of glucose of varying lengths, are found in various bacteria, yeast, fungi, and plants, and are known to be potent stimulators of the human immune system. Further, it has been shown that polysaccharides from ginseng and mushroom have active roles in immune stimulation. The ability of compositions containing purified β-glucans, ginseng extracts, and mushroom extracts to elicit immune stimulation was explored.

### Materials and Methods

“Blend A” Components

“Blend A” is a blend of purified β-glucan preparation, ginseng extracts, and mushroom extracts. Purified β-glucans from Saccharomyces cerevisiae were commercially obtained. Quality analysis determined that the commercially obtained purified β-glucan composition contained about 70% (w/w) β-glucans. Extract from Chinese ginseng (Panax ginseng C A Meyer) was also commercially obtained. Quality analysis determined that the commercially obtained ginseng extract contained about 30% (w/w) polysaccharides. Extract from princess Matsutake mushroom (Agaricus blazei Murill) was also commercially obtained. Quality analysis determined that the commercially obtained mushroom extract contained about 50% (w/w) polysaccharides.

“Blend A” Composition

Specific amounts of each of the commercially obtained purified β-glucans, ginseng extract, and mushroom extract were combined to create the “Blend A” composition. The “Blend A” composition contained, by dry weight, about 70% purified β-glucan preparation, about 20% ginseng extract, and about 10% mushroom extract.

Lymphocyte Proliferation Assay

Blood was obtained from a single donor, and peripheral blood mononuclear cells (PBMCs) were prepared by centrifugation over a ficoll hypaque density gradient, followed by washing. Cell viability and cell concentration were determined using a Beckman Coulter Vi-cell XR Cell Viability Analyzer. Cells were adjusted to a concentration of 2×10⁶/mL in complete medium, which contained RPMI 1640 medium supplemented with 25 mM HEPES buffer, 10% fetal bovine serum, 1× antibiotic/antimycotic (100 μg/mL streptomycin, 100 U/mL penicillin G, 0.25 μg/mL amphotericin B) and 50 μM 2-mercaptoethanol. PBMCs (100 μL/well) were placed in triplicate (replicates) in 96-well microtiter plates. Following this, 100 μL of complete medium, complete medium containing 1% DMSO (background controls), and five concentrations (ranging from 0.1 μg/mL-300 μg/mL) of either a purified β-glucan solution or the “Blend A” composition were added to the appropriate wells. The final DMSO concentration was kept constant at 0.5% for the DMSO-soluble compounds and the accompanying background control wells.

Cultures were incubated at 37° C. in 5% CO₂ for 3 days, pulsed with 1 μCi of tritiated thymidine (H₃-TdR) for the final 6-16 hours, harvested, and counted to determine H₃-TdR incorporation (a readout of proliferation).

Cytokine Production

PBMCs were prepared as described above and 100 μL of these cells/well were placed in triplicate in 96-well microtiter plates. Following this, 100 μL of complete medium, complete medium containing 1% DMSO (background controls), and five concentrations of either a purified β-glucan solution or the “Blend A” composition were added (see above). After approximately 24 hours of incubation at 37° C. in 5% CO₂, culture supernatants (SNs) were removed and frozen. The SNs were analyzed by multiplexing in the Luminex 200 System and assayed for the following cytokine proteins: GM-CSF, IFN-γ, IL-1β, IL-6, IL-7, IL-8, IL-10, IL-12 (p70), IL-13 and TNF-α.

### Results

The ability of the “Blend A” composition (containing β-glucans, ginseng extract, and mushroom extract) to elicit immune stimulation in comparison to purified β-glucans alone was analyzed. A detailed analysis of the components of “Blend A” and their respective polysaccharide contents is presented in Table 1. The “Blend A” composition contained fewer polysaccharides (total dry weight is about 60% polysaccharide) than the control solution of purified β-glucans alone (total dry weight is about 70% polysaccharide).

TABLE 1
“Blend A” Characteristics

| | | | | Relative Fractional |
|---|---|---|---|---|
| | | % Weight | | Contribution of |
| | | of each Extract | % Polysaccharide | Polysaccharide from |
| | % Polysaccharides | in “Blend A” | from each Extract | each Extract to Total |
| | in Extract | Composition | in “Blend A” | Polysaccharide Content |
| Extract | (dry weight) | (dry weight) | (dry weight) | of “Blend A” |
| Yeast Extract | About 70% | About 70% | About 49% | About 82% |
| (β-Glucans) | | | | |
| Ginseng Extract | About 30% | About 20% | About 6% | About 10% |
| Mushroom Extract | About 50% | About 10% | About 5% | About 8% |

Various concentrations of the purified β-glucan solution and the “Blend A” solution were added to microtiter wells housing peripheral blood mononuclear cells (PBMCs). After a period of incubation, various immunostimulation parameters and/or markers were assayed.

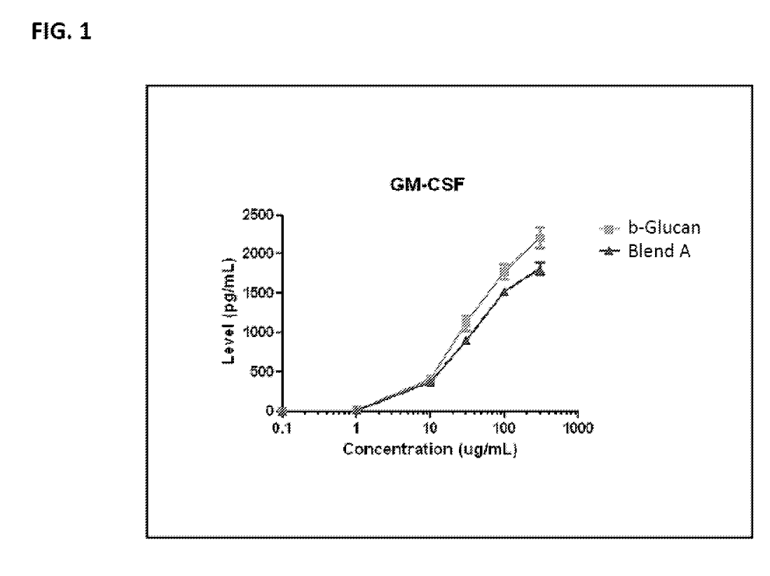

As shown in FIG. 1, it was observed that GM-CSF protein increased in response to increasing concentrations of both β-glucan solution and “Blend A” solution. However, the increase in GM-CSF protein content was greater across a range of concentrations for the β-glucan glucan solution when compared to the increase in GM-CSF observed over increasing “Blend A” concentrations. Without wishing to be bound by theory, given that it is thought that polysaccharides are main active components contributing to immune stimulation, this result could be expected as “Blend A” contained fewer total polysaccharides than the β-glucan solution.

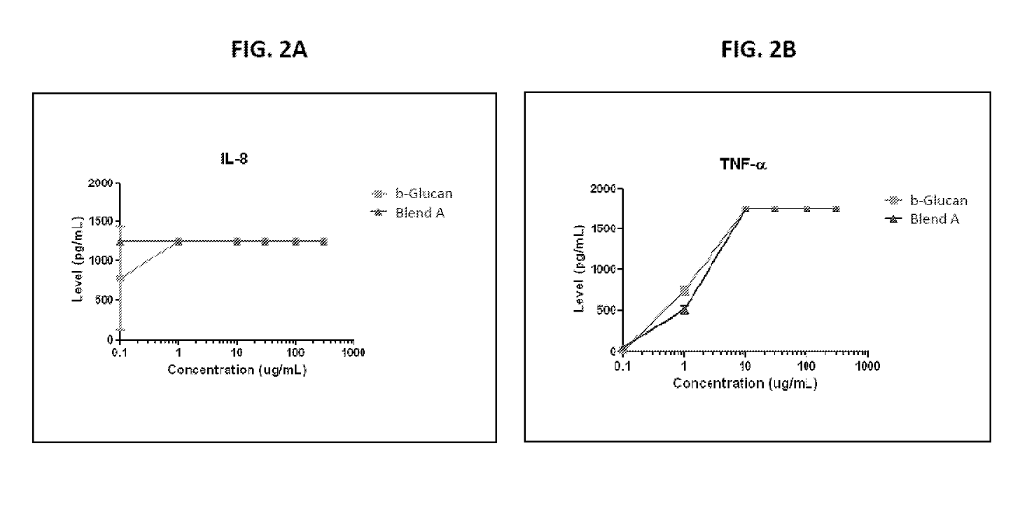

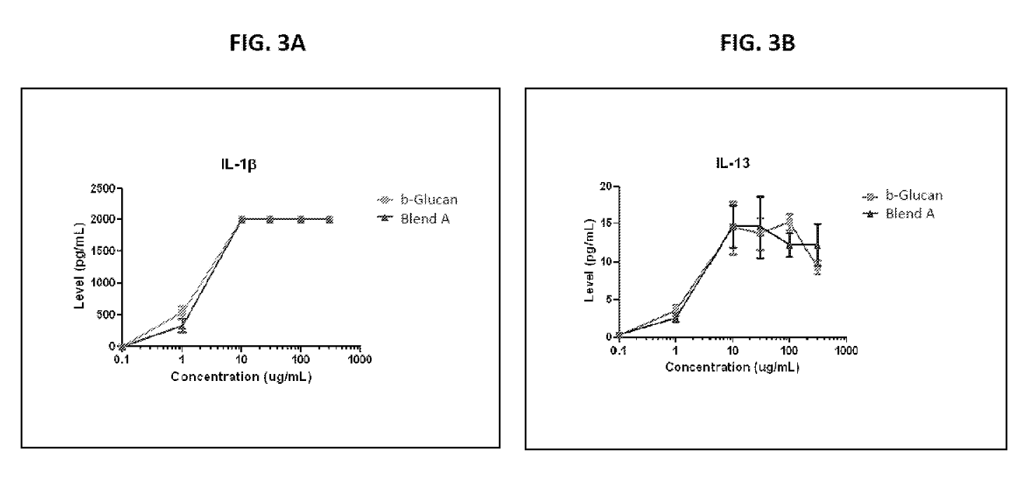

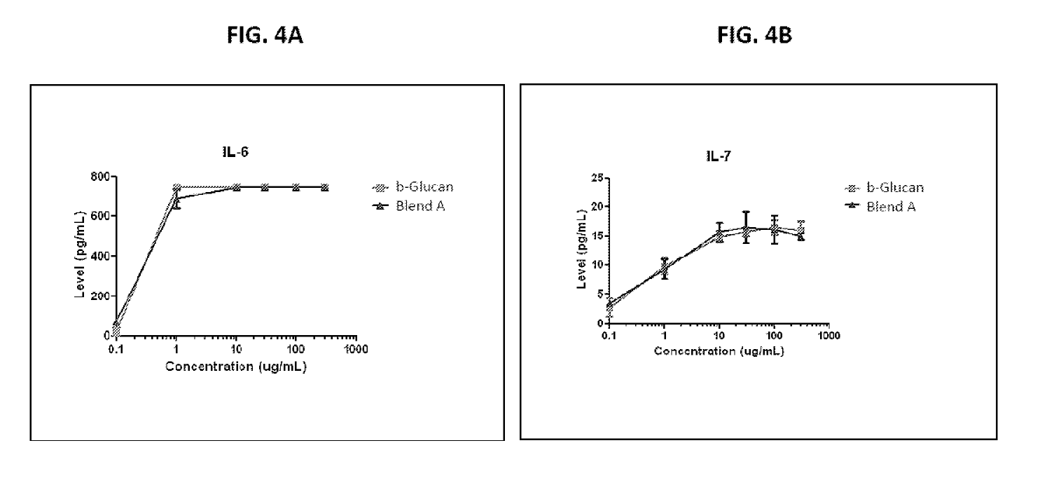

When assaying other immune stimulation markers, including IL-8 (FIG. 2A), TNF-α (FIG. 2B), IL-1β (FIG. 3A), IL-13 (FIG. 3B), IL-6 (FIG. 4A), and IL-7 (FIG. 4B), both the β-glucan solution and the “Blend A” solution elicited similar increases of these proteins across a range of concentrations. For several of these markers, the protein levels appear to plateau despite increasing concentrations of β-glucan solution or “Blend A” solution, suggesting saturation of the response. Overall, for the aforementioned markers, β-glucan solution and “Blend A” solution appear to have similar immune stimulation properties. This was intriguing, as the “Blend A” solution contains fewer total polysaccharides than the β-glucan solution yet was able to elicit similar immune stimulation.

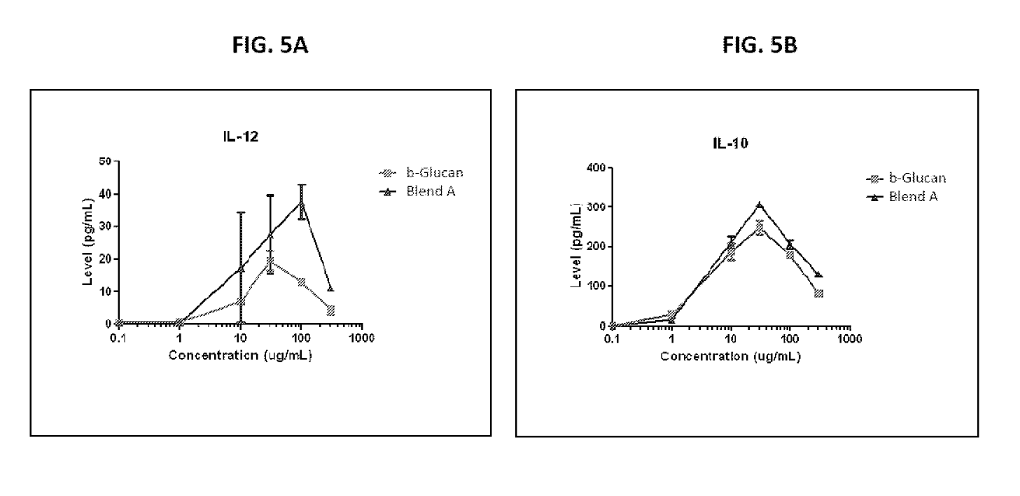

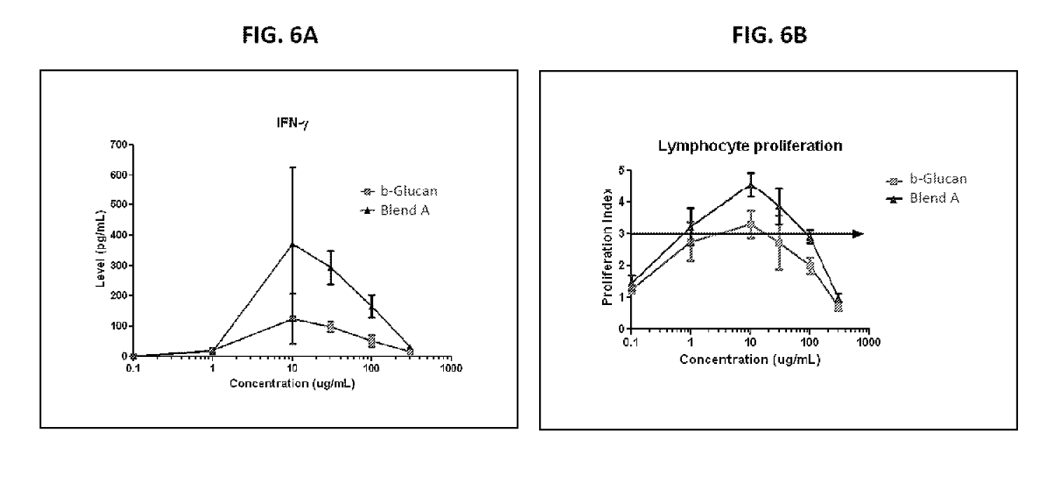

Surprisingly, it was observed that “Blend A” had a stronger immunostimulatory effect on IL-12 (FIG. 5A), IL-10 (FIG. 5B), and IFN-γ (FIG. 6A) than elicited by the β-glucan solution. This was apparent across a range of concentrations, particularly at the higher concentrations of β-glucan solution and “Blend A” tested. This was surprising; the “Blend A” composition contained fewer total polysaccharides than the β-glucan solution, but was nonetheless able to elicit stronger immune stimulation.

To explore this result further, lymphocyte proliferation in the samples in response to increasing concentrations of β-glucan solution or “Blend A” solution was tested. As shown in FIG. 6B, it was observed that “Blend A” elicited higher lymphocyte proliferation than the β-glucan glucan solution across a range of concentrations. Lymphocyte proliferation is a hallmark feature of immune stimulation, and the results demonstrate that the “Blend A” composition is a stronger stimulator of this proliferation than the β-glucan solution.

As discussed above, it was thought that the components that are active for immune stimulation from ginseng and mushroom are polysaccharides. However, the total polysaccharide content of “Blend A” (about 60% polysaccharide by dry weight) is actually less than the total polysaccharide content of the β-glucan solution (about 70% polysaccharide by dry weight). Accordingly, it was surprising that the “Blend A” composition could elicit immune stimulation to a similar degree for IL-8, TNF-α, IL-1β, II-13, IL-6, and IL-7 and a greater degree for IL-12, IL-10, IFN-γ, and lymphocyte proliferation. Different cytokines have unique roles and functions in immune systems. IFN-γ, for example, is important for enhancing the killing of cancer cells or viral infected cells by host immune cells, and this cytokine was observed to have increased stimulation after contact with “Blend A” as compared to the β-glucan solution. Further, and without wishing to be bound by theory, it is thought that the observed increase in lymphocyte proliferation after contact with “Blend A” would play an important role in enhancing both innate and adaptive immunity, including antibody production.

Overall, the totality of results position “Blend A” as being a potent immune stimulator and for having the capacity to elicit enhanced immune responses in comparison to compositions of purified polysaccharides, such as the β-glucan solution.

### Example 2: Determination/Calculation of Polysaccharide Content in “Blend A”

This Example describes the method used for determining polysaccharide content in “Blend A” using a UV/VIS-based spectrophotometric method. In this particular assay, sugars react with a reagent called anthrone reagent under acidic conditions to yield a blue-green color. The intensity of the color is measured spectrophotometrically using Dextran as a standard.

### Materials and Methods

Apparatus

Materials for the apparatus include: a water bath set to 90° C.+/−2° C.; an electronic pipettor and pipette tips; volumetric flasks of various sizes; 50 mL plastic conical centrifuge tubes, volumetric pipettes class A, glass, of various sizes; an analytical balance; a spectrophotometer HP8453 or equivalent, an electric timer, GF/C Whatman filters (>1.2 mm) or equivalent; water >16 mega ohms, 1 cm plastic cuvettes, and miscellaneous equipment as required.

Reagents

Reagents used include: Dextran powder (Sigma Catalog #D-1537 or equivalent), Anthrone (Sigma Catalog #31, 989-9 or equivalent), sulfuric acid (Fisher Catalog #A300 or equivalent), ethyl alcohol reagent anhydrous (B & J Catalog #AH090 or equivalent).

Preparation of Standard

Stock Standard Solution (1 mg/mL): 50 mg of Dextran standard was weighed into a 50 mL volume flask, dissolved and adjusted to volume with water and mix. The concentration was calculated in mg/mL. The stock standard solution was usable for 12 hours.

Working Standard Solutions: 2, 4, 6, and 8 mL of the stock standard solution were pipetted into four separately marked 200 mL volume flasks and filled to volume with water and mixed. Concentrations were about 10, 20, 30, and 40 μg/mL.

Preparation of Solutions

Anthrone Solution: 200 mg+/−1 mg of Anthrone was weighed into a 100 mL flask and filled to volume with sulfuric acid. The solution was shaken lightly to dissolve.

80:20 Ethanol/Water Solution: 800 mL of ethanol and 200 mL of water were added to a 1-liter media bottle and mixed.

### Procedure

Construction of the Standard Curve

Five 10 mL volumetric flasks were marked, four for the four working standards and one to be used as blank, and 4.0 mL of Anthrone reagent was pipetted into each respective marked flask.

The flasks were placed uncovered into an ice bath. The flasks were allowed to sit for 5-7 minutes, shaken gently, and allowed to sit an additional 5-7 minutes. Ice bath level was slightly above solution level in the flasks.

Using an electronic pipettor, 2.0 mL each of the four working standards were pipetted into respective marked flasks and 2.0 mL of water was pipetted into the one marked as blank while in the ice bath. Flasks were allowed to sit in the water bath an additional 5-7 minutes. During this time, the working standard solutions and blank formed a layer above the Anthrone reagent.

After five minutes, each flask was shaken gently to mix the layers, then covered with foil and placed into a 20 mL scintillation vial rack.

This rack was placed in a 90° C.+/−2° C. water bath for 20 minutes+/−10 seconds. A doughnut weight was used to keep the rack stable in the water bath. Hot water bath level was slightly above solution level in the flasks.

After 20 minutes, the working standards and blank were removed from the hot water bath and placed into the ice bath to cool the samples to room temperature (1.5 min. to 2 min.).

The working standards were analyzed immediately following the zeroing of the instrument using the blank. 1 cm cuvettes were used to read absorbance at 625 nm.

A standard curve was constructed by plotting the absorbance readings against the standard concentrations and the slope, Y-intercept, and the linear regression coefficient were calculated. The R² value was about 0.990.

Sample Preparations

About 120 mg of powdered “Blend A” was weighed into a 50 mL conical centrifuge tube. 50 mL of 80:20 ethanol/water was added using a volumetric pipette, vortexed briefly, and then samples were sonicated at room temperature for 1 hour. This upfront extraction is designed to remove any excipients or low molecular weight sugars from the sample matrix. The sample was centrifuged at 6000 rpm for 20 minutes at about 20° C.+/−1° C. The supernatant was immediately decanted while being extremely careful not to allow any precipitate material to be lost in the decanting process. The supernatant was discarded and the precipitate kept for polysaccharide testing.

50 mL of water was added to each centrifuge tube with the precipitate using a volumetric pipette, vortexed briefly, and then sonicated for 5-7 minutes at room temperature. Samples were centrifuged once again at 6000 rpm for 20 minutes. About 20 mL of sample was filtered through a GF/C Whatman filter into an empty tube. The resulting filtered supernatant was used for polysaccharide testing.

8.0 mL of filtered “Blend A” solution was pipetted into a 50-mL volumetric flask. The solution was filled to volume with water and mixed.

Sample Testing

4.0 mL of Anthrone reagent was pipetted into respective 10 mL volumetric flasks and the same steps above under “Construction of the Standard Curve” was followed, except performing the steps with the experimental samples. Absorbance readings should be within the 0.13-0.77 AU range.

Calculation

The total polysaccharide content (Dextran equivalent) was calculated based on the standard curve constructed as described above and using the following formula:

Total polysaccharides (% w/w as Dextran):=(A−b)×V3×V1×100/S×1000×V2×SW,

Where: A=Absorbance reading of the sample, b=intercept, SW=sample weight in milligrams, S=slope, V1=Initial Volume=50 mL, V2=Volume from V1 to dilute, V3=Final volume=50 mL

### REFERENCES

Morris, D. L. 1948, “Quantitative Determination of Carbohydrates With Dreywood's Anthrone Reagent.” Science, Mar. 5, 1948, Vol. 107.
Dreywood, R. 1946. Ind. Eng. Chem. (Anal. Ed.), 1946, 18, 499.

### Example 3: Evaluation of Immune Stimulation Elicited by Immunostimulatory Compositions

The Example demonstrates that various immunostimulatory compositions (containing β-glucans, ginseng extracts, and mushroom extracts) act as potent immune stimulators. Many of these compositions were able to stimulate increased lymphocyte proliferation and increased accumulation of multiple immunostimulation protein markers as compared to a composition containing β-glucans.

### Introduction

From Example 1, Applicants demonstrated that the solution called “Blend A” acted as a potent immune stimulator. Applicants explored the ability of other immunostimulatory compositions containing various combinations of β-glucans, ginseng extract, mushroom extract, and Astragalus extract to enhance immune responses. The results of these investigations are presented in this Example.

### Materials and Methods

Immunostimulatory Composition Components

Various immunostimulatory compositions were prepared that contain a blend of various combinations of purified β-glucan preparation, ginseng extract, mushroom extract, and Astragalus extract. Individual composition components were obtained and prepared as described in Example 1. Purified β-glucans from Saccharomyces cerevisiae were commercially obtained. Extracts from Chinese ginseng (Panax ginseng C A Meyer) or American ginseng were also commercially obtained. Extracts from princess Matsutake mushroom (Agaricus blazei Murill) or Reishi mushroom were also commercially obtained. Extracts from Astragalus were commercially obtained.

Lymphocyte Proliferation Assay

Blood was obtained from a single donor, and peripheral blood mononuclear cells (PBMCs) were prepared by centrifugation over a ficoll hypaque density gradient, followed by washing. Cell viability and cell concentration were determined using a Beckman Coulter Vi-cell XR Cell Viability Analyzer. Cells were adjusted to a concentration of 2×10⁶/mL in complete medium, which contained RPMI 1640 medium supplemented with 25 mM HEPES buffer, 10% fetal bovine serum, 1× antibiotic/antimycotic (100 μg/mL streptomycin, 100 U/mL penicillin G, 0.25 μg/mL amphotericin B) and 50 μM 2-mercaptoethanol. PBMCs (100 μL/well) were placed in triplicate (replicates) in 96-well microtiter plates. Following this, 100 μL of complete medium, complete medium containing 1% DMSO (background controls), and 10 μg/mL of either a purified β-glucan composition or the various immunostimulatory compositions were added to the appropriate wells. The final DMSO concentration was kept constant at 0.5% for the DMSO-soluble compounds and the accompanying background control wells.

Cultures were incubated at 37° C. in 5% CO₂ for 3 days, pulsed with 1 μCi of tritiated thymidine (H₃-TdR) for the final 6-16 hours, harvested, and counted to determine H₃-TdR incorporation (a readout of proliferation).

Cytokine Production

PBMCs were prepared as described above and 100 μL of these cells/well were placed in triplicate in 96-well microtiter plates. Following this, 100 μL of complete medium, complete medium containing 1% DMSO (background controls), and 10 μg/mL of either a purified β-glucan glucan composition or the various immunostimulatory compositions were added (see above). After approximately 24 hours of incubation at 37° C. in 5% CO₂, culture supernatants (SNs) were removed and frozen. The SNs were analyzed by multiplexing in the Luminex 200 System and assayed for the following cytokine proteins: GM-CSF, IFN-γ, IL-1β, IL-2, IL-4, IL-5, IL-6, IL-7, IL-8, IL-10, IL-12 (p70), IL-13 and TNF-α.

### Results

The ability of various immunostimulatory compositions (containing various combinations of β-glucans, ginseng extract, mushroom extract, and Astragalus extract) to elicit immune stimulation in comparison to purified β-glucans alone was analyzed. A detailed analysis of the contents of these compositions is presented in Table 2.

TABLE 2
Immunostimulatory Composition Characteristics

| | % Weight of each Extract in Composition (dry weight) | | | | | |
|---|---|---|---|---|---|---|
| | | | | Princess | | |
| Immuno- | Yeast | Chinese | American | Matsutake | Reishi | |
| stimulatory | Extract | Ginseng | Ginseng | mushroom | mushroom | Astragalus |
| Composition | (β-Glucans) | extract | extract | extract | extract | extract |
| SKL-P03 | 40% | 30% | 0% | 20% | 0% | 10% |
| SKL-P04 | 40% | 15% | 0% | 5% | 0% | 40% |
| SKL-P05 | 40% | 0% | 15% | 0% | 5% | 40% |
| SKL-P06 | 80% | 19% | 0% | 1% | 0% | 0% |
| SKL-P07 | 70% | 10% | 0% | 20% | 0% | 0% |
| SKL-P08 | 70% | 0% | 20% | 10% | 0% | 0% |
| SKL-P09 | 70% | 20% | 0% | 0% | 10% | 0% |
| SKL-P10 | 70% | 0% | 20% | 0% | 10% | 0% |
| SKL-B01b | 100% | 0% | 0% | 0% | 0% | 0% |

The polysaccharide content of the extracts described in Table 2 is provided below in Table 3.

TABLE 3
Polysaccharide Content of Extracts
% Polysaccharides in Extract (dry weight)

| | | | Princess | | |
|---|---|---|---|---|---|
| | Chinese | American | Matsutake | Reishi | |
| Yeast Extract | Ginseng | Ginseng | mushroom | mushroom | Astragalus |
| (β-Glucans) | extract | extract | extract | extract | extract |
| About 70% | About 30% | About 20% | About 50% | About 40% | About 60% |
| (70.3%) | (31.81%) | (19.7%) | (50.6%) | (38.1%) | (56.4%, 61.7%) |

10 μg/mL of a purified β-glucan composition (SKL-B01b) and the various immunostimulatory compositions (also 10 μg/mL) were separately added to microtiter wells housing peripheral blood mononuclear cells (PBMCs). After a period of incubation, various immunostimulation parameters and/or markers were assayed. Results were compared to a background control (containing DMSO) to account for background levels.

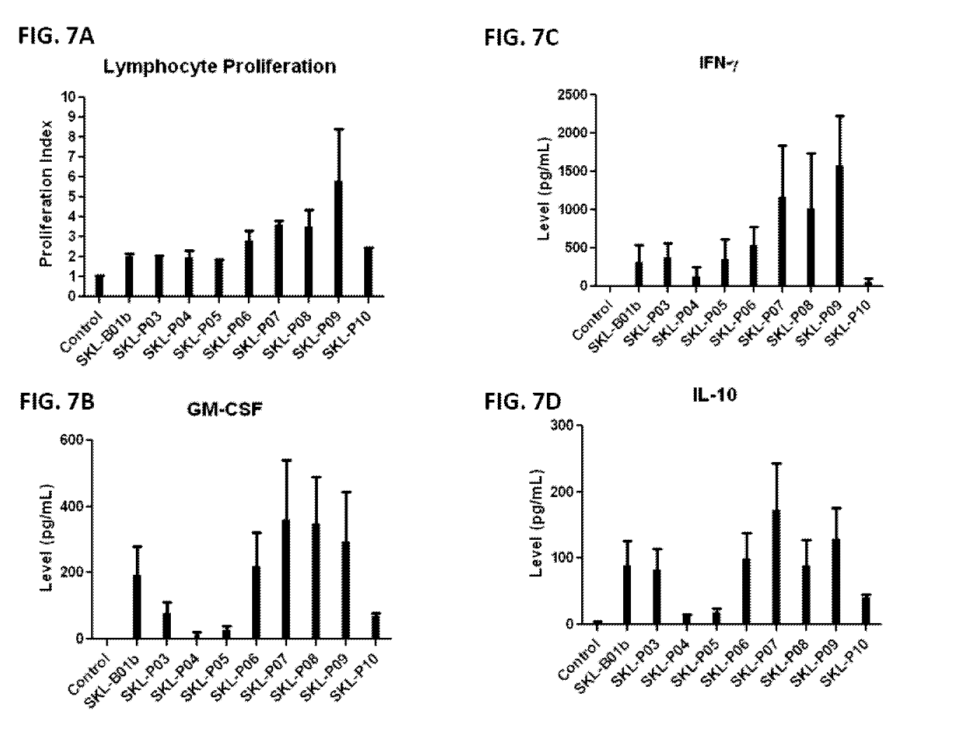

As shown in FIG. 7A, it was observed that several of the immunostimulatory compositions tested, including SKL-P06, SKL-P07, SKL-P08, and SKL-P09, elicited higher lymphocyte proliferation than the β-glucan composition (SKL-B01b) at the concentration tested (10 μg/mL). Lymphocyte proliferation is a hallmark feature of immune stimulation, and the results demonstrate that several of the compositions tested are stronger stimulators of this proliferation than the β-glucan composition. The results for the SKL-P09 composition are particularly noteworthy, as this composition exhibited the highest proliferation index.

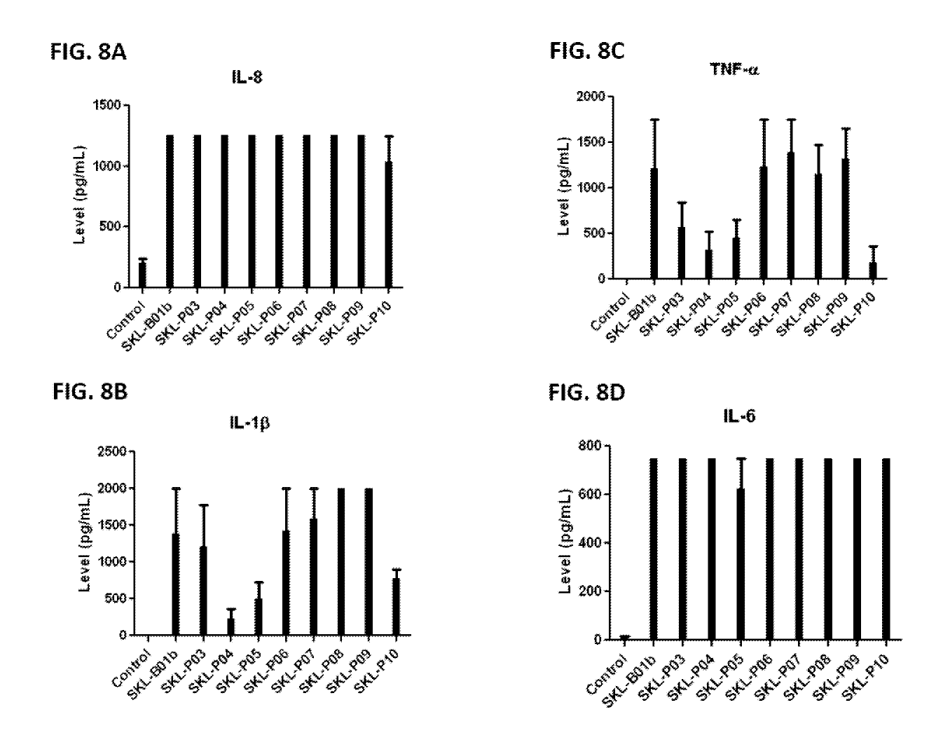

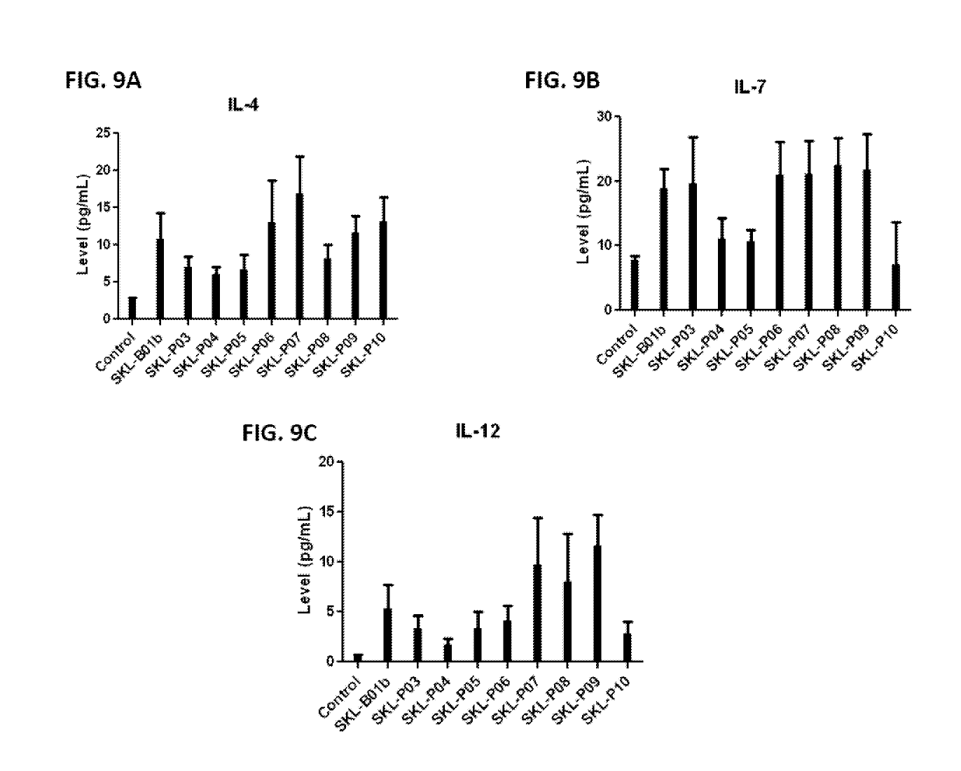

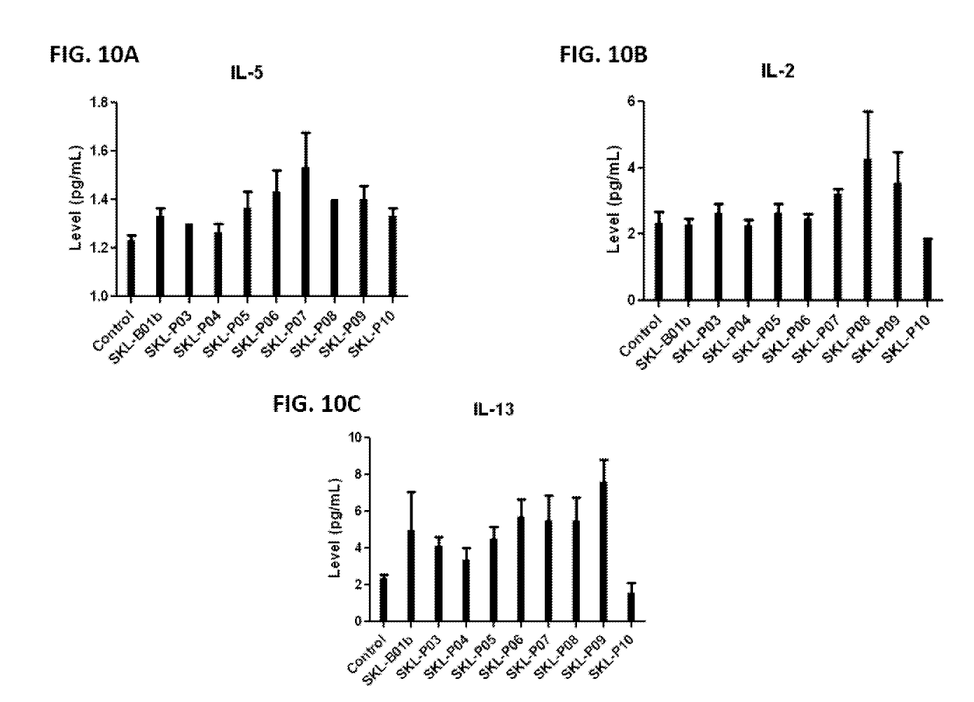

A number of immune stimulation markers were also measured, including GM-CSF (FIG. 7B), IFN-γ (FIG. 7C), IL-10 (FIG. 7D), IL-8 (FIG. 8A), IL-1β (FIG. 8B), TNF-α (FIG. 8C), IL-6 (FIG. 8D), IL-4 (FIG. 9A), IL-7 (FIG. 9B), IL-12 (FIG. 9C), IL-5 (FIG. 10A), IL-2 (FIG. 10B), and IL-13 (FIG. 10C). Overall, the data suggests that the SKL-P06, SKL-P07, SKL-P08, and SKL-P09 compositions were the most effective at inducing increases in immune stimulation markers relative to the β-glucan composition. In many instances, one or more of these compositions tended to induce increased levels of immune stimulation markers relative to the β-glucan composition (See e.g. FIG. 7B, FIG. 7C, FIG. 7D, FIG. 8B, FIG. 9A, FIG. 9C, FIG. 10A, FIG. 10B, and FIG. 10C). A similar trend was also observed for these compositions with respect to increases in lymphocyte proliferation (FIG. 7A).

Interestingly, the compositions containing Astragalus extract (SKL-P03, SKL-P04, and SKL-P05) tended to have either little to no impact on lymphocyte proliferation and immune marker stimulation relative to the β-glucan composition, or these compositions had a negative impact on these various immunostimulation parameters and/or markers relative to the β-glucan composition (See e.g. FIG. 7B, FIG. 8C, FIG. 9A, FIG. 9C, and FIG. 10C). In particular, the SKL-P04 and SKL-P05 compositions both reduced the levels of GM-CSF and IL-10 to just above background levels, and far below the levels observed with the β-glucan composition (See e.g. FIG. 7B and FIG. 7D). Overall, the data indicates that the compositions containing Astragalus extract were not strongly immunostimulatory.

Further, although the SKL-P10 composition did not contain any Astragalus extract, this composition also did not exhibit strong immunostimulatory properties, and was frequently less immunostimulatory than the β-glucan composition (See e.g. FIG. 7B, FIG. 7C, FIG. 7D, FIG. 8B, FIG. 8C, FIG. 9B, FIG. 9C, FIG. 10B, and FIG. 10C). The SKL-P10 composition contained both extract from American ginseng and extract from Reishi mushroom. Overall, the data indicates that the compositions containing both extract from American ginseng and extract from Reishi mushroom were not strongly immunostimulatory.

Overall, the totality of results position several of the compositions tested herein, such as SKL-P06, SKL-P07, SKL-P08, and SKL-P09, as being potent immune stimulators and as having the capacity to elicit enhanced immune responses in comparison to other immunostimulatory compositions, such as the β-glucan composition.

What is claimed is:

1. An immunostimulatory composition comprising a β-glucan preparation, a ginseng extract, and a mushroom extract;
wherein the β-glucan preparation is present in the range of about 40% to about 80% of the total dry weight of the β-glucan preparation, the ginseng extract, and the mushroom extract in the composition;
wherein the ginseng extract is present in the range of about 10% to about 30% of the total dry weight of the β-glucan preparation, the ginseng extract, and the mushroom extract in the composition; and
wherein the mushroom extract is present in the range of about 1% to about 20% of the total dry weight of the β-glucan preparation, the ginseng extract, and the mushroom extract in the composition.

2. The composition of claim 1, wherein the β-glucan preparation is prepared from yeast extract.

3. The composition of claim 2, wherein the yeast extract is obtained from Saccharomyces cerevisiae.

4. The composition of any one of claims 1-3, wherein the β-glucan preparation is present at about 60% to about 80% of the total dry weight of the β-glucan preparation, the ginseng extract, and the mushroom extract.

5. The composition of any one of claims 1-4, wherein the β-glucan preparation is present at about 65% to about 70% of the total dry weight of the β-glucan preparation, the ginseng extract, and the mushroom extract.

6. The composition of any one of claims 1-5, wherein the concentration of β-glucan in the β-glucan preparation is in the range of about 60% to about 80% of the total dry weight of the β-glucan preparation.

7. The composition of claim 6, wherein the concentration of β-glucan in the β-glucan preparation is about 60% to about 75% of the total dry weight of the preparation.

8. The composition of claim 6, wherein the concentration of β-glucan in the β-glucan preparation is at least about 60%, about 65%, about 70%, or about 80% of the total dry weight of the preparation.

9. The composition of any one of claims 1-8, wherein the ginseng extract is prepared from Chinese ginseng or American ginseng.

10. The composition of any one of claims 1-9, wherein the ginseng extract is present at about 20% to about 30% of the total dry weight of the β-glucan preparation, the ginseng extract, and the mushroom extract.

11. The composition of any one of claims 1-10, wherein the ginseng extract is present at about 20% to about 25% of the total dry weight of the β-glucan preparation, the ginseng extract, and the mushroom extract.

12. The composition of any one of claims 1-10, wherein the ginseng extract is present at about 20%, about 25%, or about 30% of the total dry weight of the β-glucan preparation, the ginseng extract, and the mushroom extract.

13. The composition of any one of claims 1-12, wherein the concentration of polysaccharides in the ginseng extract is about 5% to about 50% of the total dry weight of the ginseng extract.

14. The composition of claim 13, wherein the concentration of polysaccharides in the ginseng extract is about 10% to about 45% polysaccharides of the total dry weight of the ginseng extract.

15. The composition of claim 13, wherein the concentration of polysaccharides in the ginseng extract is about 20% to about 35% of total dry weight of the ginseng extract.

16. The composition of any one of claims 1-15, wherein the mushroom extract is prepared from a mushroom selected from the group consisting of princess Matsutake mushroom, Reishi mushroom, shitake mushroom, Enokie mushroom, and pine mushroom.

17. The composition of any one of claims 1-16, wherein the mushroom extract is present at about 5% to about 15% of the total dry weight of the β-glucan preparation, the ginseng extract, and the mushroom extract.

18. The composition of any one of claims 1-17, wherein the mushroom extract is present at about 5% to about 10% of the total dry weight of the β-glucan preparation, the ginseng extract, and the mushroom extract.

19. The composition of any one of claims 1-16, wherein the mushroom extract is about 5%, about 7.5%, about 10%, or about 15% of the total dry weight of the β-glucan preparation, the ginseng extract, and the mushroom extract.

20. The composition of any one of claims 1-19, wherein the concentration of polysaccharides in the mushroom extract is about 30% to about 55% of the total dry weight of the mushroom extract.

21. The composition of claim 20, wherein the concentration of polysaccharides in the mushroom extract is about 30%, about 35%, about 40%, about 45%, about 50%, or about 55% of the total dry weight of the mushroom extract.

22. The composition of any one of claims 1-21, wherein the β-glucan preparation is present at about 65% to about 70% of the total dry weight of the β-glucan preparation, the ginseng extract, and the mushroom extract; the ginseng extract is from Chinese ginseng and is present at about 20% to about 25% of the total dry weight of the β-glucan preparation, the ginseng extract, and the mushroom extract, and; the mushroom extract is from princess Matsutake mushroom and is present at about 5% to about 10% of the total dry weight of the β-glucan preparation, the ginseng extract, and the mushroom extract.

23. The composition of any one of claims 1-21, wherein the β-glucan preparation is present at about 75% to about 80% of the total dry weight of the β-glucan preparation, the ginseng extract, and the mushroom extract; the ginseng extract is from Chinese ginseng and is present at about 15% to about 20% of the total dry weight of the β-glucan preparation, the ginseng extract, and the mushroom extract, and; the mushroom extract is from princess Matsutake mushroom and is present at about 1% to about 5% of the total dry weight of the β-glucan preparation, the ginseng extract, and the mushroom extract.

24. The composition of any one of claims 1-21, wherein the β-glucan preparation is present at about 65% to about 70% of the total dry weight of the β-glucan preparation, the ginseng extract, and the mushroom extract; the ginseng extract is from Chinese ginseng and is present at about 10% to about 15% of the total dry weight of the β-glucan preparation, the ginseng extract, and the mushroom extract, and; the mushroom extract is from princess Matsutake mushroom and is present at about 15% to about 20% of the total dry weight of the β-glucan preparation, the ginseng extract, and the mushroom extract.

25. The composition of any one of claims 1-21, wherein the β-glucan preparation is present at about 65% to about 70% of the total dry weight of the β-glucan preparation, the ginseng extract, and the mushroom extract; the ginseng extract is from American ginseng and is present at about 20% to about 25% of the total dry weight of the β-glucan preparation, the ginseng extract, and the mushroom extract, and; the mushroom extract is from princess Matsutake mushroom and is present at about 5% to about 10% of the total dry weight of the β-glucan preparation, the ginseng extract, and the mushroom extract.

26. The composition of any one of claims 1-21, wherein the β-glucan preparation is present at about 65% to about 70% of the total dry weight of the β-glucan preparation, the ginseng extract, and the mushroom extract; the ginseng extract is from Chinese ginseng and is present at about 20% to about 25% of the total dry weight of the β-glucan preparation, the ginseng extract, and the mushroom extract, and; the mushroom extract is from Reishi mushroom and is present at about 5% to about 10% of the total dry weight of the β-glucan preparation, the ginseng extract, and the mushroom extract.

27. An orally consumable product comprising the composition of any one of claims 1-26.

28. The product of claim 27, wherein the orally consumable product is selected from the group consisting of a foodstuff composition, a beverage product, a dietary supplement, a nutraceutical, an edible gel mix, an edible gel composition, a granulated powder, a soft gel composition, a flash dissolve composition, and a pharmaceutical composition.

29. The product of claim 27, wherein the orally consumable product is formulated into a tablet or capsule.

30. The product of claim 27, wherein the orally consumable product is a foodstuff composition selected from the group consisting of a confectionary composition, a condiment, a cereal composition, a baked good, and a dairy product.

31. The product of claim 27, wherein the orally consumable product is a carbonated or non-carbonated beverage product.

32. The product of claim 27, wherein the orally consumable product is a beverage product selected from the group consisting of a soft drink, a fountain beverage, a frozen and ready-to-drink beverage, coffee, tea, a dairy beverage, a powdered soft drink, a liquid concentrate, fruit juice, a sport drink, and an energy drink.

33. The product of any one of claims 27-32, further comprising one or more additives selected from the group consisting of a carbohydrate, a polyol, an amino acid or salt thereof, a poly-amino acid or salt thereof, a sugar acid or salt thereof, a nucleotide, an organic acid, an inorganic acid, an organic salt, an organic acid salt, an organic base salt, an inorganic salt, a bitter compound, a flavorant, a flavoring ingredient, an astringent compound, a protein, a protein hydrolysate, a surfactant, an emulsifier, a flavonoid, an alcohol, a polymer, and combinations thereof.

34. A method of preparing a composition, the method comprising combining a β-glucan preparation, a ginseng extract, and a mushroom extract into a composition, wherein the composition is formulated such that the β-glucan preparation is present in the range of about 40% to about 80% of the total dry weight of the β-glucan preparation, the ginseng extract, and the mushroom extract in the composition, the ginseng extract is present in the range of about 10% to about 30% of the total dry weight of the β-glucan preparation, the ginseng extract, and the mushroom extract in the composition, and the mushroom extract is present in the range of about 1% to about 20% of the total dry weight of the β-glucan preparation, the ginseng extract, and the mushroom extract in the composition.

35. The method of claim 34, wherein the β-glucan preparation is prepared from yeast extract.

36. The method of claim 35, wherein the yeast extract is obtained from Saccharomyces cerevisiae.

37. The method of any one of claims 34-36, wherein the β-glucan preparation is present at about 60% to about 80% of the total dry weight of the β-glucan preparation, the ginseng extract, and the mushroom extract.

38. The method of any one of claims 34-37, wherein the β-glucan preparation is present at about 65% to about 70% of the total dry weight of the β-glucan preparation, the ginseng extract, and the mushroom extract.

39. The method of any one of claims 34-38, wherein the concentration of β-glucan in the β-glucan glucan preparation is in the range of about 60% to about 80% of the total dry weight of the preparation.

40. The method of claim 39, wherein the concentration of β-glucan in the β-glucan preparation is about 60% to about 75% of the total dry weight of the preparation.

41. The method of claim 39, wherein the concentration of β-glucan in the β-glucan preparation is at least about 60%, about 65%, about 70%, or about 80% of the total dry weight of the preparation.

42. The method of any one of claims 34-41, wherein the ginseng extract is prepared from Chinese ginseng or American ginseng.

43. The method of any one of claims 34-42, wherein the ginseng extract is present at about 20% to about 30% of the total dry weight of the β-glucan preparation, the ginseng extract, and the mushroom extract.

44. The method of any one of claims 34-43, wherein the ginseng extract is present at about 20% to about 25% of the total dry weight of the β-glucan preparation, the ginseng extract, and the mushroom extract.

45. The method of any one of claims 34-43, wherein the ginseng extract is about 20%, about 25%, or about 30% of the total dry weight of the β-glucan preparation, the ginseng extract, and the mushroom extract.

46. The method of any one of claims 34-45, wherein the concentration of polysaccharides in the ginseng extract is about 5% to about 50% of the total dry weight of the ginseng extract.

47. The method of claim 46, wherein the concentration of polysaccharides in the ginseng extract is about 10% to about 45% polysaccharides of the total dry weight of the ginseng extract.

48. The method of claim 46, wherein the concentration of polysaccharides in the ginseng extract is about 20% to about 35% of total dry weight of the ginseng extract.

49. The method of any one of claims 34-48, wherein the mushroom extract is prepared from a mushroom selected from the group consisting of princess Matsutake mushroom, Reishi mushroom, shitake mushroom, Enokie mushroom, and pine mushroom.

50. The method of any one of claims 34-49, wherein the mushroom extract is present at about 5% to about 15% of the total dry weight of the β-glucan preparation, the ginseng extract, and the mushroom extract.

51. The method of any one of claims 34-50, wherein the mushroom extract is present at about 5% to about 10% of the total dry weight of the β-glucan preparation, the ginseng extract, and the mushroom extract.

52. The method of any one of claims 34-50, wherein the mushroom extract is about 5%, about 7.5%, about 10%, or about 15% of the total dry weight of the β-glucan preparation, the ginseng extract, and the mushroom extract.

53. The method of any one of claims 34-52, wherein the concentration of polysaccharides in the mushroom extract is about 30% to about 55% of the total dry weight of the mushroom extract.

54. The method of claim 53, wherein the concentration of polysaccharides in the mushroom extract is about 30%, about 35%, about 40%, about 45%, or about 50% of the total dry weight of the mushroom extract.

55. The method of any one of claims 34-54, wherein the β-glucan preparation is present at about 65% to about 70% of the total dry weight of the β-glucan preparation, the ginseng extract, and the mushroom extract; the ginseng extract is from Chinese ginseng and is present at about 20% to about 25% of the total dry weight of the β-glucan preparation, the ginseng extract, and the mushroom extract, and; the mushroom extract is from princess Matsutake mushroom and is present at about 5% to about 10% of the total dry weight of the β-glucan preparation, the ginseng extract, and the mushroom extract.

56. The method of any one of claims 34-54, wherein the β-glucan preparation is present at about 75% to about 80% of the total dry weight of the β-glucan preparation, the ginseng extract, and the mushroom extract; the ginseng extract is from Chinese ginseng and is present at about 15% to about 20% of the total dry weight of the β-glucan preparation, the ginseng extract, and the mushroom extract, and; the mushroom extract is from princess Matsutake mushroom and is present at about 1% to about 5% of the total dry weight of the β-glucan preparation, the ginseng extract, and the mushroom extract.

57. The method of any one of claims 34-54, wherein the β-glucan preparation is present at about 65% to about 70% of the total dry weight of the β-glucan preparation, the ginseng extract, and the mushroom extract; the ginseng extract is from Chinese ginseng and is present at about 10% to about 15% of the total dry weight of the β-glucan preparation, the ginseng extract, and the mushroom extract, and; the mushroom extract is from princess Matsutake mushroom and is present at about 15% to about 20% of the total dry weight of the β-glucan preparation, the ginseng extract, and the mushroom extract.

58. The method of any one of claims 34-54, wherein the β-glucan preparation is present at about 65% to about 70% of the total dry weight of the β-glucan preparation, the ginseng extract, and the mushroom extract; the ginseng extract is from American ginseng and is present at about 20% to about 25% of the total dry weight of the β-glucan preparation, the ginseng extract, and the mushroom extract, and; the mushroom extract is from princess Matsutake mushroom and is present at about 5% to about 10% of the total dry weight of the β-glucan preparation, the ginseng extract, and the mushroom extract.

59. The method of any one of claims 34-54, wherein the β-glucan preparation is present at about 65% to about 70% of the total dry weight of the β-glucan preparation, the ginseng extract, and the mushroom extract; the ginseng extract is from Chinese ginseng and is present at about 20% to about 25% of the total dry weight of the β-glucan preparation, the ginseng extract, and the mushroom extract, and; the mushroom extract is from Reishi mushroom and is present at about 5% to about 10% of the total dry weight of the β-glucan glucan preparation, the ginseng extract, and the mushroom extract.

60. The method of any one of claims 34-59, further comprising combining one or more additives into the composition.

61. The method of any one of claims 34-60, further comprising preparing the composition into an orally consumable product.

62. A method of enhancing an immune function in a subject, the method comprising administering to a subject the composition of any one of claims 1-26 or the product of any one of claims 27-33.

63. The method of claim 62, wherein the subject is an animal.

64. The method of claim 63, wherein the subject is a human.

65. The method of any one of claims 62-64, wherein the enhanced immune function comprises enhanced lymphocyte proliferation and/or enhanced production of one or more cytokines.

66. The method of claim 65, wherein the one or more cytokines are selected from the group consisting of IFN-γ, IL-12, and IL-10.

67. A kit for enhancing immune function in a subject, the kit comprising the immunostimulatory composition of any one of claims 1-26 or the product of any one of claims 27-33.

68. The kit of claim 67, wherein the kit comprises instructions for administering the immunostimulatory composition to the subject.

69. A method of enhancing an immune function in a subject, the method comprising administering to a subject a composition comprising about 220 mg to about 250 mg of a β-glucan preparation, about 75 to about 90 mg of a ginseng extract, and about 20 mg to about 40 mg of a mushroom extract, wherein the composition is administered once daily.

70. The method of claim 69, wherein the composition comprises about 220 mg, about 230 mg, about 240 mg, or about 250 mg of a β-glucan preparation.

71. The method of any one of claim 69 or 70, wherein the composition comprises about 80 mg, about 82 mg, about 84 mg, about 86 mg, about 88 mg, or about 90 mg of a ginseng extract.

72. The method of any one of claims 69-71, wherein the composition comprises about 20 mg, about 25 mg, about 30 mg, about 33 mg, about 35 mg, about 38 mg, or about 40 mg of a mushroom extract.

73. The method of any one of claims 69-72, wherein the composition comprises about 220 mg, about 230 mg, about 240 mg, or about 250 mg of a β-glucan preparation; about 80 mg, about 82 mg, about 84 mg, about 86 mg, about 88 mg, or about 90 mg of a ginseng extract; and about 20 mg, about 25 mg, about 30 mg, about 33 mg, about 35 mg, about 38 mg, or about 40 mg of a mushroom extract.

74. A method of enhancing an immune function in a subject, the method comprising administering to a subject a composition comprising about 250 mg to about 325 mg of a β-glucan preparation, about 90 to about 120 mg of a ginseng extract, and about 30 mg to about 50 mg of a mushroom extract, wherein the composition is administered once daily.

75. The method of claim 74, wherein the composition comprises about 250 mg, about 265 mg, about 275 mg, about 290 mg, about 300 mg, about 310 mg, or about 325 mg of a β-glucan preparation.

76. The method of any one of claim 74 or 75, wherein the composition comprises about 90 mg, about 95 mg, about 100 mg, about 105 mg, about 110 mg, about 115 mg, or about 120 mg of a ginseng extract.

77. The method of any one of claims 74-76, wherein the composition comprises about 30 mg, about 35 mg, about 40 mg, about 45 mg, or about 50 mg of a mushroom extract.

78. The method of any one of claims 74-77, wherein the composition comprises about 250 mg, about 265 mg, about 275 mg, about 290 mg, about 300 mg, about 310 mg, or about 325 mg of a β-glucan preparation; about 90 mg, about 95 mg, about 100 mg, about 105 mg, about 110 mg, about 115 mg, or about 120 mg of a ginseng extract of a ginseng extract; and about 30 mg, about 35 mg, about 40 mg, about 45 mg, or about 50 mg of a mushroom extract.

79. A method of enhancing an immune function in a subject, the method comprising administering to a subject a composition comprising about 150 mg to about 200 mg β-glucans from a β-glucan preparation, about 5 mg to about 10 mg polysaccharides from a ginseng extract, and about 8 mg to about 16 mg polysaccharides from a mushroom extract, wherein the composition is administered once daily.

80. The method of claim 79, wherein the composition comprises about 150 mg β-glucans, about 175 mg β-glucans, or about 200 mg β-glucans from a β-glucan preparation.

81. The method of any one of claim 79 or 80, wherein the composition comprises about 5 mg, about 6 mg, about 7 mg, about 8 mg, about 9 mg, or about 10 mg polysaccharides from a ginseng extract.

82. The method of any one of claims 79-81, wherein the composition comprises about 8 mg, about 9 mg, about 10 mg, about 11 mg, about 12 mg, about 13 mg, about 14 mg, about 15 mg, or about 16 mg polysaccharides from a mushroom extract.

83. The method of any one of claims 79-82, wherein the composition comprises about 150 mg β-glucans, about 175 mg β-glucans, or about 200 mg β-glucans from a β-glucan preparation; about 5 mg, about 6 mg, about 7 mg, about 8 mg, about 9 mg, or about 10 mg polysaccharides from a ginseng extract; and about 8 mg, about 9 mg, about 10 mg, about 11 mg, about 12 mg, about 13 mg, about 14 mg, about 15 mg, or about 16 mg polysaccharides from a mushroom extract.

84. A method of enhancing an immune function in a subject, the method comprising administering to a subject a composition comprising about 150 mg to about 200 mg β-glucans from a β-glucan preparation, about 20 mg to about 30 mg polysaccharides from a ginseng extract, and about 10 mg to about 20 mg polysaccharides from a mushroom extract, wherein the composition is administered once daily.

85. The method of claim 84, wherein the composition comprises about 150 mg β-glucans, about 175 mg β-glucans, or about 200 mg β-glucans from a β-glucan preparation.

86. The method of any one of claim 84 or 85, wherein the composition comprises about 20 mg, about 25 mg, or about 30 mg polysaccharides from a ginseng extract.

87. The method of any one of claims 84-86, wherein the composition comprises about 10 mg, about 12 mg, about 14 mg, about 16 mg, about 18 mg, or about 20 mg polysaccharides from a mushroom extract.

88. The method of any one of claims 84-87, wherein the composition comprises about 150 mg β-glucans, about 175 mg β-glucans, or about 200 mg β-glucans from a β-glucan preparation; about 20 mg, about 25 mg, or about 30 mg polysaccharides from a ginseng extract; and about 10 mg, about 12 mg, about 14 mg, about 16 mg, about 18 mg, or about 20 mg polysaccharides from a mushroom extract.

89. A method of enhancing an immune function in a subject, the method comprising administering to a subject a composition comprising about 175 mg to about 225 mg β-glucans from a β-glucan preparation, about 25 mg to about 45 mg polysaccharides from a ginseng extract, and about 15 mg to about 30 mg polysaccharides from a mushroom extract, wherein the composition is administered once daily.

90. The method of claim 89, wherein the composition comprises about 175 mg β-glucans, about 195 mg β-glucans, about 200 mg β-glucans, about 205 mg β-glucans, about 210 mg β-glucans, about 215 mg β-glucans, or about 225 mg β-glucans from a β-glucan preparation.

91. The method of any one of claim 89 or 90, wherein the composition comprises about 25 mg, about 28 mg, about 30 mg, about 32 mg, about 35 mg, about 38 mg, about 40 mg, about 42 mg, or about 45 mg polysaccharides from a ginseng extract.

92. The method of any one of claims 89-91, wherein the composition comprises about 15 mg, about 18 mg, about 20 mg, about 22 mg, about 24 mg, about 25 mg, or about 30 mg polysaccharides from a mushroom extract.

93. The method of any one of claims 89-92, wherein the composition comprises about 175 mg β-glucans, about 195 mg β-glucans, about 200 mg β-glucans, about 205 mg β-glucans, about 210 mg β-glucans, about 215 mg β-glucans, or about 225 mg β-glucans from a β-glucan preparation; about 25 mg, about 28 mg, about 30 mg, about 32 mg, about 35 mg, about 38 mg, about 40 mg, about 42 mg, or about 45 mg polysaccharides from a ginseng extract; and about 15 mg, about 18 mg, about 20 mg, about 22 mg, about 24 mg, about 25 mg, or about 30 mg polysaccharides from a mushroom extract.
